(19) United States
(12) Patent Application Publication
(10) Pub. No.: US 2022/0228201 A1
(43) Pub. Date: Jul. 21, 2022

(54) MOLECULAR ARRAYS AND METHODS FOR GENERATING AND USING THE ARRAYS

(71) Applicant: 10x Genomics, Inc., Pleasanton, CA (US)

(72) Inventors: Geoffrey MCDERMOTT, Livermore, CA (US); David Michael PATTERSON, Oakland, CA (US); Andrew D. PRICE, San Diego, CA (US); Eswar Prasad RAMACHANDRAN IYER, Sunnyvale, CA (US); Michael SCHNALL-LEVIN, San Francisco, CA (US)

(21) Appl. No.: 17/565,028

(22) Filed: Dec. 29, 2021

Related U.S. Application Data
(60) Provisional application No. 63/132,379, filed on Dec. 30, 2020.

(51) Int. Cl. C12Q 1/6837; C12Q 1/6869

(52) U.S. Cl. CPC C12Q 1/6837; C12Q 1/6869

(57) ABSTRACT

Provided in some aspects are methods for light-controlled in situ surface patterning of a substrate. Compositions such as nucleic acid arrays produced by the methods are also disclosed. In some embodiments, provided herein is photocontrollable hybridization, where oligonucleotides with photo-caged bases prevent hybridization of complementary nucleic acid strands. Uncaging of the oligonucleotides allows hybridization and/or ligation of nucleic acid molecules at the exposed area. A large diversity of barcodes can be created in molecules on the substrate via sequential rounds of light exposure, hybridization, and ligation.

### CROSS-REFERENCE TO RELATED APPLICATION

This application claims priority to U.S. Provisional Patent Application No. 63/132,379, filed Dec. 30, 2020, entitled “MOLECULAR ARRAYS AND METHODS AND USES THEREOF,” which is herein incorporated by reference in its entirety for all purposes.

### FIELD

The present disclosure relates in some aspects to molecular arrays and methods for manufacturing molecular arrays in situ.

### BACKGROUND

Arrays of nucleic acids are an important tool in the biotechnology industry and related fields. These nucleic acid arrays, in which a plurality of distinct or different nucleic acids are positioned on a solid support surface in the form of an array or pattern, find use in a variety of applications, including gene expression analysis, drug screening, nucleic acid sequencing, mutation analysis (e.g., single nucleotide polymorphisms (SNPs) and/or copy number variations (CNV)), and the like.

A feature of many arrays that have been developed is that each of the distinct nucleic acids of the array is stably attached to a discrete location on the array surface, such that its position remains constant and known throughout the use of the array. Stable attachment is achieved in a number of different ways, including covalent bonding of a nucleic acid polymer to the support surface and non-covalent interaction of the nucleic acid polymer with the surface.

There are two main ways of producing nucleic acid arrays in which the immobilized nucleic acids are covalently attached to the substrate surface, i.e., via in situ synthesis in which the nucleic acid polymer is grown on the surface of the substrate in a step-wise, nucleotide-by-nucleotide fashion, or via deposition of a full length, presynthesized nucleic acid/polypeptide, cDNA fragment, etc., onto the surface of the array.

While nucleic acid arrays have been manufactured using in situ synthesis techniques, applications in the field of genomics and high throughput screening have fueled the demand for precise chemistry and high fidelity of the synthesized oligonucleotides. Accordingly, there is continued interest in the development of new methods for producing nucleic acid arrays in situ. Provided herein are methods, uses and articles of manufacture that meet such needs.

### SUMMARY

In some aspects, provided herein is a method for providing an array of polynucleotides, comprising: irradiating a first polynucleotide immobilized on a substrate with a first light while a second polynucleotide immobilized on the substrate is not irradiated with the first light, wherein the first polynucleotide comprises a first photo-cleavable moiety that inhibits or blocks hybridization and/or ligation to the first polynucleotide, and the second polynucleotide comprises a second photo-cleavable moiety that inhibits or blocks hybridization and/or ligation to the second polynucleotide, thereby cleaving the first photo-cleavable moiety such that the inhibition or blocking of hybridization and/or ligation to the first polynucleotide is reduced or eliminated, whereas hybridization and/or ligation to the second polynucleotide remains inhibited or blocked by the second photo-cleavable moiety, wherein a first oligonucleotide of at least four nucleotide residues in length is attached to the first polynucleotide via hybridization and/or ligation, thereby providing on the substrate an array comprising the first and second polynucleotides. In some embodiments, the first polynucleotide is ligated to the first oligonucleotide or a portion thereof and the second polynucleotide is not ligated to the first oligonucleotide or portion thereof. In some embodiments, the first oligonucleotide or portion thereof comprises a barcode region comprising one or more barcode sequences, and the first polynucleotide is barcoded with the one or more barcode sequences and the second polynucleotide is not barcoded with the one or more barcode sequences.

In some aspects, provided herein is a method for providing an array of polynucleotides, comprising: irradiating a first polynucleotide immobilized on a substrate with a first light while a second polynucleotide immobilized on the substrate is not irradiated with the first light, wherein the first polynucleotide comprises a first photo-cleavable moiety that inhibits or blocks hybridization and/or ligation to the first polynucleotide, and the second polynucleotide comprises a second photo-cleavable moiety that inhibits or blocks hybridization and/or ligation to the second polynucleotide, thereby cleaving the first photo-cleavable moiety such that the inhibition or blocking of hybridization and/or ligation to the first polynucleotide is reduced or eliminated, whereas hybridization and/or ligation to the second polynucleotide remains inhibited or blocked by the second photo-cleavable moiety, wherein a first barcode is attached to the first polynucleotide via hybridization and/or ligation, thereby providing on the substrate an array comprising the first and second polynucleotides, wherein the first polynucleotide is barcoded with the first barcode and the second polynucleotide is not barcoded with the first barcode.

In some aspects, provided herein is method for providing an array of polynucleotides, wherein a first polynucleotide immobilized on a substrate is irradiated with a first light while a second polynucleotide immobilized on the substrate is not irradiated with the first light, wherein the first polynucleotide comprises a first photo-cleavable moiety that inhibits or blocks hybridization and/or ligation to the first polynucleotide, and the second polynucleotide comprises a second photo-cleavable moiety that inhibits or blocks hybridization and/or ligation to the second polynucleotide, and the first photo-cleavable moiety is cleaved such that the inhibition or blocking of hybridization and/or ligation to the first polynucleotide is reduced or eliminated, whereas hybridization and/or ligation to the second polynucleotide remains inhibited or blocked by the second photo-cleavable moiety, the method comprising attaching a first barcode to the first polynucleotide via hybridization and/or ligation, thereby providing on the substrate an array comprising the first and second polynucleotides, wherein the first polynucleotide is barcoded with the first barcode and the second polynucleotide is not barcoded with the first barcode.

In any of the embodiments herein, the second polynucleotide can be irradiated with a second light, and wherein the second photo-cleavable moiety is cleaved such that the inhibition or blocking of hybridization and/or ligation to the second polynucleotide is reduced or eliminated.

In any of the embodiments herein, the method can further comprise irradiating the second polynucleotide with a second light, thereby cleaving the second photo-cleavable moiety such that the inhibition or blocking of hybridization and/or ligation to the second polynucleotide is reduced or eliminated.

In any of the embodiments herein, the second polynucleotide can be irradiated with the second light while the first polynucleotide is not irradiated with the second light.

In any of the embodiments herein, a second oligonucleotide of at least four nucleotide residues in length can be attached to the second polynucleotide via hybridization and/or ligation, thereby providing on the substrate an array comprising the first and second polynucleotides. In some embodiments, the second polynucleotide is ligated to the second oligonucleotide or a portion thereof and the first polynucleotide is not ligated to the second oligonucleotide or portion thereof. In some embodiments, the second oligonucleotide or portion thereof comprises a barcode region comprising one or more barcode sequences, and the second polynucleotide is barcoded with the one or more barcode sequences of the second oligonucleotide or portion thereof and the first polynucleotide is not barcoded with the one or more barcode sequences of the second oligonucleotide or portion thereof, wherein the first polynucleotide is barcoded with the one or more barcode sequences of the first oligonucleotide or portion thereof and the second polynucleotide is not barcoded with the one or more barcode sequences of the first oligonucleotide or portion thereof.

In any of the embodiments herein, a second barcode can be attached to the second polynucleotide via hybridization and/or ligation, wherein an array comprising the first and second polynucleotides is provided on the substrate, and wherein the first polynucleotide is barcoded with the first barcode and the second polynucleotide is barcoded with the second barcode.

In any of the embodiments herein, the method can further comprise attaching a second barcode to the second polynucleotide via hybridization and/or ligation, thereby providing on the substrate an array comprising the first polynucleotide barcoded with the first barcode and the second polynucleotide barcoded with the second barcode.

In one embodiment, disclosed herein is a method for providing an array of polynucleotides, comprising: (a) irradiating a first polynucleotide immobilized on a substrate with a first light while a second polynucleotide immobilized on the substrate is not irradiated with the first light, wherein the first polynucleotide comprises a first photo-cleavable moiety that inhibits or blocks hybridization and/or ligation to the first polynucleotide, and the second polynucleotide comprises a second photo-cleavable moiety that inhibits or blocks hybridization and/or ligation to the second polynucleotide, thereby cleaving the first photo-cleavable moiety such that the inhibition or blocking of hybridization and/or ligation to the first polynucleotide is reduced or eliminated, whereas hybridization and/or ligation to the second polynucleotide remains inhibited or blocked by the second photo-cleavable moiety, and (b) attaching a first barcode to the first polynucleotide via hybridization and/or ligation, thereby providing on the substrate an array comprising the first and second polynucleotides, wherein the first polynucleotide is barcoded with the first barcode and the second polynucleotide is not barcoded with the first barcode.

In some aspects, the method can further comprise: (c) irradiating the second polynucleotide with a second light, thereby cleaving the second photo-cleavable moiety such that the inhibition or blocking of hybridization and/or ligation to the second polynucleotide is reduced or eliminated. In any of the embodiments herein, the second polynucleotide can be irradiated with the second light while the first polynucleotide is not irradiated with the second light. In any of the embodiments herein, the method can further comprise (d) attaching a second barcode to the second polynucleotide via hybridization and/or ligation, thereby providing on the substrate an array comprising the first polynucleotide barcoded with the first barcode and the second polynucleotide barcoded with the second barcode.

In any of the embodiments herein, the first polynucleotide and the second polynucleotide can be of the same nucleic acid sequence or different nucleic acid sequences.

In any of the embodiments herein, the first polynucleotide and/or the second polynucleotide can be single stranded or double stranded.

In any of the embodiments herein, the first and second polynucleotides on the substrate can comprise one or more common sequences. In any of the embodiments herein, the one or more common sequences can comprise a homopolymeric sequence, such as a poly(dT) sequence, of three, four, five, six, seven, eight, nine, ten or more nucleotide residues in length. In any of the embodiments herein, the one or more common sequences can comprise a common primer sequence. In some embodiments, the common primer sequence is between about 10 and about 35 nucleotides in length. In any of the embodiments herein, the one or more common sequences can comprise a partial primer sequence. For example, a terminal sequence of a polynucleotide on the substrate together with a sequence of an oligonucleotide attached to the polynucleotide molecule on the substrate can form the hybridization sequence for a primer. In this example, the terminal sequence of the polynucleotide on the substrate can be viewed as a partial primer sequence. In any of the embodiments herein, substrate prior to the irradiating step, the first and second polynucleotides on the substrate can be identical in sequence. In any of the embodiments herein, the first and second polynucleotides on the substrate can be different in sequences, optionally the first and second polynucleotides on the substrate can comprise different barcode sequences.

In any of the embodiments herein, the first and second polynucleotides on the substrate can be immobilized in a plurality of features. In any of the embodiments herein, the 3′ terminal nucleotides of the first and second polynucleotides on the substrate can be distal to the substrate. In any of the embodiments herein, the 5′ terminal nucleotides of the first and second polynucleotides on the substrate can be more proximal to the substrate than the 3′ terminal nucleotides. In any of the embodiments herein, one or more nucleotides at or near the 5′ terminus of each of the first and second polynucleotides on the substrate can be directly or indirectly attached to the substrate. In any of the embodiments herein, the 3′ terminus of each of the first and second polynucleotides on the substrate can project away from the substrate. In any of the embodiments herein, the 5′ terminal nucleotides of the first and second polynucleotides on the substrate can be distal to the substrate. In any of the embodiments herein, the 3′ terminal nucleotides of the first and second polynucleotides on the substrate can be more proximal to the substrate than the 5′ terminal nucleotides. In any of the embodiments herein, one or more nucleotides at or near the 3′ terminus of each of the first and second polynucleotides on the substrate can be directly or indirectly attached to the substrate. In any of the embodiments herein, the 5′ terminus of each of the first and second polynucleotides on the substrate can project away from the substrate.

In any of the embodiments herein, the first and second polynucleotides on the substrate prior to the irradiating step can be between about 4 and about 100 nucleotides in length. In any of the embodiments herein, the first and second polynucleotides on the substrate prior to the irradiating step can be between about 10 and about 50 nucleotides in length.

In any of the embodiments herein, the first polynucleotide and/or the second polynucleotide can be DNA oligonucleotides. In any of the embodiments herein, the first polynucleotide and/or the second polynucleotide can be between about 6 and about 30 nucleotides in length. In some embodiments, the first polynucleotide and/or the second polynucleotide can be between about 10 and about 20 nucleotides in length.

In any of the embodiments herein, the first and second polynucleotides on the substrate can be part of an array comprising an arrangement of a plurality of features, e.g., each comprising one or more molecules such as a nucleic acid molecule (e.g., a DNA oligo). In some embodiments, the array comprises different oligonucleotides in different features. In some embodiments, oligonucleotide molecules on the substrate are immobilized in a plurality of features. Nucleotides immobilized on the substrate may be of different orientations. For example, in some embodiments, the 3′ terminal nucleotides of immobilized oligonucleotide molecules are distal to the substrate. In some embodiments, the 5′ terminal nucleotides of immobilized oligonucleotide molecules are distal to the substrate. In embodiments, where 5′ terminal nucleotides of immobilized oligonucleotides are distal to the substrate, capping can involve blocking the 5′ termini, for example via incorporation of a modified nucleotide (e.g., 7-methylguanine). The oligonucleotide molecules on the substrate prior to the irradiating step may have a variety of properties, which include but are not limited to, length, orientation, structure, and modifications. The oligonucleotide molecules on the substrate prior to the irradiating step can be of about 5, about 6, about 7, about 8, about 9, about 10, about 15, about 20, about 25, about 30, about 35, about 40, about 45, about 50, about 55, about 60, about 70, about 80, about 90, or about 100 nucleotides in length. In some embodiments, oligonucleotide molecules on the substrate prior to the irradiating step are between about 5 and about 50 nucleotides in length. The oligonucleotide molecules on the substrate may comprise functional groups. In some embodiments, the functional groups are amino or hydroxyl groups.

In any of the embodiments herein, the first light and the second light can be the same, or can be different. In any of the embodiments herein, the first light and the second light can be of different wavelengths, intensities, or durations of irradiation, or any combination thereof.

In any of the embodiments herein, hybridization and/or ligation to the first polynucleotide barcoded with the first barcode can be inhibited or blocked, and/or hybridization and/or ligation to the second polynucleotide barcoded with the second barcode can be inhibited or blocked.

In any of the embodiments herein, the first barcode can comprise a third photo-cleavable moiety that that inhibits or blocks hybridization and/or ligation, thereby inhibiting or blocking hybridization and/or ligation to the first polynucleotide barcoded with the first barcode, and/or the second barcode can comprise a fourth photo-cleavable moiety that that inhibits or blocks hybridization and/or ligation, thereby inhibiting or blocking hybridization and/or ligation to the second polynucleotide barcoded with the second barcode.

In any of the embodiments herein, the first photo-cleavable moiety, the second photo-cleavable moiety, the third photo-cleavable moiety, and/or the fourth photo-cleavable moiety can be the same or different.

In any of the embodiments herein, the first photo-cleavable moiety, the second photo-cleavable moiety, the third photo-cleavable moiety, and/or the fourth photo-cleavable moiety can inhibit or block hybridization.

In any of the embodiments herein, the first photo-cleavable moiety, the second photo-cleavable moiety, the third photo-cleavable moiety, and/or the fourth photo-cleavable moiety can inhibit or block ligation.

In any of the embodiments herein, the first photo-cleavable moiety, the second photo-cleavable moiety, the third photo-cleavable moiety, and/or the fourth photo-cleavable moiety can comprise a photo-caged nucleobase.

In any of the embodiments herein, the first photo-cleavable moiety, the second photo-cleavable moiety, the third photo-cleavable moiety, and/or the fourth photo-cleavable moiety can comprise a photo-cleavable hairpin.

In any of the embodiments herein, the first photo-cleavable moiety, the second photo-cleavable moiety, the third photo-cleavable moiety, and/or the fourth photo-cleavable moiety can comprise a photo-caged functional group, such as a photo-caged hydroxyl group (e.g., 3-hydroxyl group), a photo-caged amino group, a photo-caged aldehyde group, and/or a photo-caged click chemistry group, optionally wherein the click chemistry group is capable of a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, or a Cu(I)-catalyzed azide-alkyne cycloaddition (CuAAC) reaction.

In any of the embodiments herein, the first photo-cleavable moiety, the second photo-cleavable moiety, the third photo-cleavable moiety, and/or the fourth photo-cleavable moiety can comprise a photo-cleavable polymer.

In any of the embodiments herein, the first barcode and the second barcode can be of the same nucleic acid sequence or different nucleic acid sequences.

In any of the embodiments herein, the first barcode and the second barcode can be single stranded or double stranded.

In any of the embodiments herein, the first barcode and the second barcode can be DNA oligonucleotides.

In any of the embodiments herein, the first barcode and the second barcode can independently be between about 4 and about 50 nucleotides in length. In any of the embodiments herein, the first barcode and the second barcode can independently be between about 5 and about 25 nucleotides in length. In any of the embodiments herein, the first barcode and the second barcode can independently be between about 5 and about 20 nucleotides in length. In some embodiments, the first barcode and the second barcode can independently be between about 6 and about 16 nucleotides in length.

In any of the embodiments herein, the substrate can comprise a plurality of differentially barcoded polynucleotides immobilized thereon.

In any of the embodiments herein, the substrate can be transparent, translucent, or opaque.

In any of the embodiments herein, the irradiation can comprise using a photomask to selectively irradiate the first polynucleotide or the second polynucleotide.

In any of the embodiments herein, the attachment of the first barcode and/or the second barcode can comprise ligating one end of the first/second barcode to one end of the first/second polynucleotide, respectively.

In any of the embodiments herein, the 5′ end nucleotide of the first/second barcode can be ligated to the 3′ end nucleotide of the first/second polynucleotide, respectively, or the 3′ end nucleotide of the first/second barcode can be ligated to the 5′ end nucleotide of the first/second polynucleotide, respectively. In some embodiments, the 3′ end nucleotide of the first/second polynucleotide is the 3′ end nucleotide remaining after removal of a photo-cleavable moiety from the respective polynucleotide. In some embodiments, the 5′ end nucleotide of the first/second polynucleotide is the 5′ end nucleotide remaining after removal of a photo-cleavable moiety from the respective polynucleotide.

In any of the embodiments herein, the attachment of the first barcode can comprise hybridizing one end of the first barcode and one end of the first polynucleotide to a first splint, and/or the attachment of the second barcode can comprise hybridizing one end of the second barcode and one end of the second polynucleotide to a second splint.

In any of the embodiments herein, the method further comprises ligating the first barcode to the first polynucleotide hybridized to the first splint, and/or ligating the second barcode to the second polynucleotide hybridized to the second splint. In some embodiments, the first/second barcode is directly ligated to the first/second polynucleotide, respectively, without gap filling. In some embodiments, ligating the first/second barcode to the first/second polynucleotide, respectively, is preceded by gap filling.

In any of the embodiments herein, the first splint and the second splint can be of the same nucleic acid sequence or different nucleic acid sequences.

In any of the embodiments herein, the first splint and/or the second splint can be single stranded.

In any of the embodiments herein, the first splint and/or the second splint can be DNA oligonucleotides, and the first splint and/or the second splint can be at least 4, 5, 6, 7, 8, 9, 10, 11, 12, 13, 14, or 15 nucleotides in length.

In any of the embodiments herein, the method can further comprise removing the first splint and/or the second splint after the ligation. In any of the embodiments herein, the first splint and/or the second splint can be removed by heat and/or treatment with a denaturing agent, such as KOH or NaOH.

In any of the embodiments herein, the method can further comprise providing the first polynucleotide and the second polynucleotide immobilized on the substrate.

In any of the embodiments herein, the method can further comprise blocking the 3′ or 5′ termini of barcoded polynucleotide molecules from ligation, e.g., prior to and/or during the ligation of other barcoded or nonbarcoded polynucleotide molecules to oligonucleotide molecules of at least four residues in length. In any of the embodiments herein, the blocking can comprise adding a 3′ dideoxy, a non-ligating 3′ phosphoramidate, or a triphenylmethyl (trityl) group to the barcoded polynucleotide molecules and/or unligated polynucleotide molecules, optionally wherein the blocking by the trityl group is removed with a mild acid after ligation is completed. In any of the embodiments herein, the addition can be catalyzed by a terminal transferase, e.g., TdT. In any of the embodiments herein, the blocking can be removed using an internal digestion of the barcoded polynucleotide molecules after ligation is completed.

In any of the embodiments herein, the method can comprise N cycles, wherein Nis an integer of 2 or greater, and one or more or all of the N cycles comprises the irradiating and the attaching steps. In any of the embodiments herein, the irradiating and the attaching steps can be repeated N cycles, each cycle for one or more regions of the substrate (e.g., for one or more features on an array), for a round until all desired regions have been exposed to light, deprotected once, and polynucleotide molecules in the exposed regions have received a barcode sequence for that round, which barcode sequence may be the same or different for molecules for any two given regions (e.g., features on an array). The barcode sequences for different cycles (e.g., each cycle for a different region of the substrate) in the same round can comprise the same or different sequences, and preferably the barcode sequences for different cycles are different. In any of the embodiments herein, the barcode sequences received by polynucleotide molecules in feature(s) on the substrate in cycle I and in feature(s) in cycle J can be different, wherein I and J are integers and 1≤I<J≤N.

In any of the embodiments herein, the method can comprise M rounds, wherein M is an integer of 2 or greater, and each of the M rounds comprises one or more cycles. In any of the embodiments herein, each of the M rounds may comprise N cycles, optionally wherein each cycle is for attaching oligonucleotides to polynucleotide molecules in one or more regions of the substrate (e.g., for one or more features on the array). In any of the embodiments herein, each of the M rounds can comprise N cycles, wherein Nis 3 or greater. In any of the embodiments herein, each of the M rounds can comprise the same number of cycles, or two or more of the M rounds can comprise different numbers of cycles. In some embodiments, each of Round 1 and Round M can comprise Cycle 1, Cycle 2, . . . , and Cycle N. However, it should be appreciated that in some embodiments, any two rounds of Round 1 to Round M may comprise the same number or different numbers of sequential cycles. For instance, Round 2 may comprise fewer than N cycles, whereas Round 3 may comprise more than N cycles. As an example, Cycle 1 and Cycle 2 of Round 2 may be combined into one cycle and the regions in these cycles receive the same oligonucleotide, and in Round 3 the regions after Cycle (N−1) may be grouped into two sets, one set for Cycle N and the other set for Cycle (N+1), and each set may receive a different oligonucleotide. One or more rounds comprising the attachment of a common nucleic acid sequence may be performed before or after any of Round 1 to Round M, and the nucleic acid sequence can be common to two or more regions on the substrate. In some cases, the nucleic acid sequence can be universal and can be shared by all of the regions on the substrate.

In any of the embodiments herein, polynucleotide molecules in a feature of the substrate can receive a first barcode sequence in one of the cycles in round K, wherein K is an integer and 1≤K<M, and polynucleotide molecules in the feature comprising the first barcode sequence receive a second barcode sequence in one of the cycles in round (K+1), thereby forming polynucleotide molecules comprising the first and second barcode sequences. In any of the embodiments herein, the diversity of barcode sequences in the polynucleotides in a plurality of features on the substrate can be N^{M}. In any of the embodiments herein, the feature(s) can be no more than 0.5 micron, no more than 1 micron, no more than 5 microns, no more than 7 microns, no more than 10 microns, or no more than 15 microns, no more than 20 microns, no more than 25 microns, no more than 30 microns, or no more than 35 microns, no more than 40 microns, no more than 45 microns, or no more than 50 microns in diameter. In any of the embodiments herein, the feature(s) can be no more than 500 nm, no more than 600 nm, no more than 700 nm, no more than 800 nm, no more than 900 nm, no more than 1 micron, no more than 1.5 microns, no more than 2 microns, no more than 2.5 microns, no more than 3 microns, no more than 3.5 microns, no more than 4 microns, no more than 4.5 microns, or no more than 5 microns in one dimension. In any of the embodiments herein, the feature(s) can be no more than 500 nm, no more than 600 nm, no more than 700 nm, no more than 800 nm, no more than 900 nm, no more than 1 micron, no more than 1.5 microns, no more than 2 microns, no more than 2.5 microns, no more than 3 microns, no more than 3.5 microns, no more than 4 microns, no more than 4.5 microns, or no more than 5 microns in two dimensions.

In some aspects, provided herein is a method for providing an array, comprising: (a) irradiating a substrate comprising an unmasked first region and a masked second region, whereby photo-cleavable moieties of oligonucleotide molecules in the first region are cleaved to render oligonucleotide molecules in the first region available for hybridization and/or ligation, whereas oligonucleotide molecules in the second region are protected by photo-cleavable moieties of oligonucleotide molecules in the second region from hybridization and/or ligation; and (b) attaching a first oligonucleotide of at least four residues in length (e.g., comprising a first barcode sequence) to oligonucleotide molecules in the first region via hybridization and/or ligation, wherein oligonucleotide molecules in the second region are not ligated to the first oligonucleotide or a portion thereof, thereby providing on the substrate an array comprising different oligonucleotide molecules in the first and second regions. In some aspects, the method further comprises (a′) irradiating the unmasked second region, whereby photo-cleavable moieties of oligonucleotide molecules in the second region are cleaved to render oligonucleotide molecules in the second region available for hybridization and/or ligation; and (b′) attaching a second oligonucleotide of at least four residues in length (e.g., comprising a second barcode sequence) to oligonucleotide molecules in the second region via hybridization and/or ligation, whereas oligonucleotide molecules in the first region are not hybridized and/or ligated to the second oligonucleotide. For instance, oligonucleotide molecules in the first region may be protected by photo-cleavable moieties of oligonucleotide molecules in the first region from hybridization and/or ligation, and/or splints can be used to hybridize to the second oligonucleotide and template ligation of the second oligonucleotide specifically to oligonucleotide molecules in the second region but not to oligonucleotide molecules in the first region based on sequence complementarity.

In some aspects, provided herein is a composition comprising: (i) a substrate comprising a first region and a second region, (ii) hybridization complexes in the first region, wherein at least one of the hybridization complexes comprise a polynucleotide molecule (e.g., a first polynucleotide) immobilized in the first region hybridized to a first splint, which is in turn hybridized to a first oligonucleotide comprising a first barcode sequence, and (iii) polynucleotide molecules immobilized in the second region and protected by a photo-cleavable moiety that inhibits or blocks hybridization and/or ligation to the polynucleotide molecules immobilized in the second region.

In some embodiments, provided herein is a composition, comprising: (i) a substrate comprising a first region and a second region, (ii) hybridization complexes in the first region, wherein at least one of the hybridization complexes comprise a polynucleotide molecule immobilized in the first region hybridized to a first splint, which is in turn hybridized to a first oligonucleotide comprising a first barcode sequence, wherein the hybridization complexes are protected by a first photo-cleavable moiety that inhibits or blocks hybridization and/or ligation, and (iii) polynucleotide molecules immobilized in the second region and protected by a second photo-cleavable moiety that inhibits or blocks hybridization and/or ligation. In some embodiments, the first photo-cleavable moiety and the second photo-cleavable moiety are the same. In some embodiments, the first and second photo-cleavable moieties are different. In any of the embodiments herein, the polynucleotide molecules on the substrate can comprise functional groups, optionally wherein the functional groups can be amino or hydroxyl groups. In some embodiments, the functional groups can be 3′ hydroxy groups of nucleotides. In any of the embodiments herein, the functional groups have been reacted with and/or protected by a photo-sensitive group, moiety, or molecule to form a photo-cleavable moiety.

In some embodiments, provided herein is a composition, comprising a substrate comprising a plurality of universal polynucleotide molecules immobilized thereon, wherein the universal polynucleotide molecules have been reacted with and/or protected by a photo-sensitive group, moiety, or molecule to form a photo-cleavable moiety and protected from hybridization and/or ligation. In some embodiments, the composition further comprises a photomask masking a second region while exposing a first region of the substrate to light. In some embodiments, the composition comprises hybridization complexes in the first region, wherein at least one of the hybridization complexes comprise a universal polynucleotide molecule immobilized in the first region hybridized to a first splint, which is in turn hybridized to a first oligonucleotide comprising a first barcode sequence. In some embodiments, the universal polynucleotide molecules on the substrate can comprise functional groups. In some embodiments, the functional groups may be amino or hydroxyl groups. In some embodiments, the functional groups may be deprotected by removing or degrading a photo-sensitive group, moiety, or molecule. In some embodiments, the functional groups can be 3′ hydroxyl group of nucleotides.

In any of the embodiments herein, the composition can further comprise a ligase capable of ligating the first oligonucleotide and the polynucleotide molecule immobilized in the first region using the first splint as template, and optionally a polymerase capable of gap filling using the first splint as template prior to the ligation. In any of the embodiments herein, the composition may not comprise any dNTP or a polymerase capable of incorporating a dNTP into an oligonucleotide molecule. In any of the embodiments herein, the composition may not comprise any reagent for base-by-base oligonucleotide synthesis.

In any of the embodiments herein, a method disclosed herein may not comprises a step of contacting the substrate or polynucleotide molecules immobilized thereon with any reagent for base-by-base oligonucleotide synthesis.

### BRIEF DESCRIPTION OF THE DRAWINGS

FIG. 1 shows an exemplary light-controlled method of patterning a surface in situ for producing an array on the surface, wherein certain nucleic acid molecules are barcoded while certain other nucleic acid molecules are not.

FIG. 2 shows an exemplary light-controlled method of patterning a surface in situ for producing an array on the surface, wherein certain nucleic acid molecules are barcoded with different barcodes.

FIG. 3 shows another exemplary light-controlled method of patterning a surface in situ for producing an array on the surface, wherein certain nucleic acid molecules are barcoded with different barcodes.

FIG. 4 shows another exemplary light-controlled method of patterning a surface in situ, wherein cycles and/or rounds of light-controlled barcoding (e.g., via hybridization followed by splinted ligation) may be repeated to reach a desired barcode diversity.

FIG. 5 shows examples of photo-cleavable groups that can block hybridization or ligation.

FIG. 6 shows an exemplary method of attaching a barcode (BC1) and subsequently removing a population of unprotected nucleic acid molecules (P3) that have not received the barcode.

FIG. 7 shows an exemplary method of removing a population of nucleic acid molecules (P3) that have not received a barcode (BC1) and subsequent attachment of another barcode (BC2) to the correct nucleic acid molecule (P2).

FIG. 8 shows an exemplary method comprising attaching a barcode (BC1), removing unprotected nucleic acid molecules that have not received the barcode, and subsequently attaching another barcode (BC2) to the correct nucleic acid molecule (P2).

FIG. 9 shows an exemplary method of removing nucleic acid molecules (P2 and P3) that have not received a barcode (BC1) and subsequent attachment of an adaptor (U) prior to attaching the next barcode (not shown) to nucleic acid molecules (P1) that have received the correct barcode (BC1).

FIG. 10 shows an example of pre-patterning a substrate prior to cycles of light-controlled surface patterning.

FIG. 11 shows an example of light-controlled surface patterning on a pre-patterned array.

FIG. 12 shows an exemplary barcode assembly scheme for use in light-controlled surface patterning.

FIG. 13 provides quantitative polymerase chain reaction (qPCR) results demonstrating light-controlled ligation of an oligonucleotide comprising a photoprotective group (PPG) to an immobilized base oligonucleotide.

### DETAILED DESCRIPTION

Oligonucleotide arrays for spatial transcriptomics may be made by mechanical spotting, bead arrays, and/or in situ base-by-base synthesis of the oligonucleotides. In some cases, mechanical spotting is ideal for larger spot sizes (e.g., 30 microns in diameter or greater), since fully elaborated oligonucleotides (e.g., with a desired combination and diversity of barcodes) can be spotted in a known position with high purity and fidelity. However, methods to decrease spot sizes or features at or below 10 microns (e.g., single cell scale resolution) in diameter with sufficient density are lacking. In some aspects, bead arrays offer a way to increase feature density. For example, barcodes are generated by first attaching an oligonucleotide to all beads and then performing multiple rounds of split-pool ligations to generate barcodes combinatorially. However, in some aspects, bead arrays result in random barcoded bead arrays that must be decoded prior to use and each array ultimately has a unique pattern. Additionally, even monodisperse beads at the 1-10 micron scale may have some variability that results in a range of feature sizes with the potential for variable oligonucleotide density.

Methods for base-by-base synthesis oligonucleotides on arrays have utilized photo-cleavable protecting groups to synthesize barcode oligonucleotides one nucleotide at a time. However, the base-by-base approach requires a large number of UV exposure steps and developer exposures causing the final oligonucleotide sequence to contain a large number of errors or modifications for example through depurination at various points in the synthesized oligonucleotide sequence. In addition, photo-caged oligonucleotides are expensive and is not tenable at the current price point to synthesize full-length oligonucleotides suitable for molecular assays. For example, each UV deprotecting step is not 100% efficient, meaning some caged oligonucleotides will not be available for hybridization and ligation. Over multiple rounds of the base-by-base approach this can cause a significant drop in expected surface concentration of oligos. For example, the oligonucleotide fidelity for in situ base-by-base arrays may decrease with increasing oligonucleotide length with a ˜99% per step efficiency.

Provided herein are methods and uses of light-controlled combinatorial barcode generation for in situ arrays. In some embodiments, light-controlled ligation for in situ combinatorial barcode generation is utilized.

A. Methods of Light-Controlled Surface Patterning In Situ

In some aspects, provided herein is a method of patterning a surface in situ for producing an array on the surface, for example, by spatially-selective light-activated hybridization/ligation generating DNA sequences and/or combination of DNA sequences at spatial positions in the array. In some embodiments, the diversity of the DNA sequences and/or the combinations of DNA sequences can be generated combinatorially, and the DNA sequence or combination thereof at a particular spatial location in the array can be unique compared to those at some or all other spatial locations in the array. In some embodiments, the method comprises assembling nucleic acid sequences (e.g., barcode sequences, gene sequences, or genomic sequences including non-coding sequences) on immobilized oligonucleotides, e.g., based on hybridization and/or ligation, on a slide or wafer surface. In some embodiments, the in situ method uses photo-caged hybridization/ligation to enable barcodes to be generated combinatorially, for example, in as few as three rounds of assembly. Hybridization and/or ligation of barcodes can be controlled, for example, using one or more photo-cleavable moieties. For example, hybridization can be blocked using a synthetic nucleotide with a photo-cleavable protecting group on a nucleobase and/or a photo-cleavable hairpin that dissociates upon cleavage. In other examples, ligation can be controlled using a photo-cleavable moiety, such as a photo-caged 3′-hydroxyl group.

Oligonucleotides may be immobilized on a substrate according a number of known methods, such as the methods set forth in U.S. Pat. Nos. 6,737,236, 7,259,258, 7,309,593, 7,375,234, 7,427,678, 5,610,287, 5,807,522, 5,837,860, and 5,472,881; U.S. Patent Application Publication Nos. 2008/0280773, 2011/0143967, and 2011/0059865; Shalon et al. (1996) Genome Research, 639-645; Rogers et al. (1999) Analytical Biochemistry 266, 23-30; Stimpson et al. (1995) Proc. Natl. Acad. Sci. USA 92, 6379-6383; Beattie et al. (1995) Clin. Chem. 45, 700-706; Lamture et al. (1994) Nucleic Acids Research 22, 2121-2125; Beier et al. (1999) Nucleic Acids Research 27, 1970-1977; Joos et al. (1997) Analytical Biochemistry 247, 96-101; Nikiforov et al. (1995) Analytical Biochemistry 227, 201-209; Timofeev et al. (1996) Nucleic Acids Research 24, 3142-3148; Chrisey et al. (1996) Nucleic Acids Research 24, 3031-3039; Guo et al. (1994) Nucleic Acids Research 22, 5456-5465; Running and Urdea (1990) BioTechniques 8, 276-279; Fahy et al. (1993) Nucleic Acids Research 21, 1819-1826; Zhang et al. (1991) 19, 3929-3933; and Rogers et al. (1997) Gene Therapy 4, 1387-1392. The entire contents of each of the foregoing documents are incorporated herein by reference.

In some embodiments, oligonucleotides may be immobilized by spotting (e.g., DNA printing) on a substrate with reactive surface chemistry, such as a polymer (e.g., a hydrophilic polymer) containing epoxy reactive groups. In some embodiments, the polymer comprises a passivating polymer. In some embodiments, the polymer comprises a photoreactive group for attachment to the substrate (such as a glass slide). In some embodiments, the oligonucleotides may be immobilized in a DNA printing buffer, optionally wherein the printing buffer comprises a surfactant such as sarcosyl (e.g., a buffer containing sodium phosphate and about 0.06% sarcosyl). In some embodiments, after immobilization of the oligonucleotides, one or more wash and/or blocking steps are performed. Blocking steps can comprise contacting the substrate with a solution that deactivates or blocks unreacted functional groups on the substrate surface. In one example, the blocking buffer can comprise ethanolamine (e.g., to deactivate epoxy silane or other epoxy reactive functional groups).

In some embodiments, provided herein is a method to generate an array with barcode diversity in the 100s, 1,000s, 10,000s, 100,000s, 1,000,000s, or 10,000,000s. In some embodiments, a substrate comprising a dense lawn of a common oligonucleotide bearing a photo-protected hybridization region is provided. Using a series of photomasks, oligonucleotides in desired regions of the lawn may be iteratively deprotected. In some embodiments, the method further comprises attaching a round 1 barcode to one or more deprotected oligonucleotides, for example, by attaching an oligonucleotide cassette with a complementary region (e.g., complementary to a splint), a barcode region, and a photo-cleavable moiety such as a photo-protected hybridization region. In some embodiments, the attachment may be performed by placing the substrate in a chamber or vessel (e.g., within which oligonucleotides such as those comprising barcode sequences can be delivered and ligated to nucleic acid molecules on the substrate). In some embodiments, the chamber or vessel is a flow cell or a device comprising microfluidic channels. In some embodiments, the method comprises flowing in the round 1 barcode (e.g., an oligonucleotide cassette) to be attached to the common oligonucleotide. The process can be repeated N cycles (each cycle for one or more features on an array) for round 1 until all desired features have been deprotected and the common oligonucleotides in the features have received the round 1 barcode which may be the same or different for molecules in any two given features. The round 1 barcode molecules can be ligated to the common oligonucleotides. The process can be repeated M rounds to achieve a desired barcode diversity, for example, by attaching a round 2 barcode (which may be the same or different for molecules in any two given features), a round 3 barcode (which may be the same or different for molecules in any two given features), . . . , and a round m barcode (which may be the same or different for molecules in any two given features) to each of the growing oligonucleotides in the features. In some embodiments, each round comprises a plurality of cycles (each cycle for one or more features on an array) of deprotection and oligonucleotide attachment until all desired features have been deprotected once and the molecules in the features have received the barcode(s) (which may be the same or different for molecules in any two given features) for that round. In some embodiments, all or a subpopulation of the barcoded oligonucleotides are deprotected, e.g., by exposure to light. In some embodiments, the method further comprises attaching a capture sequence to the deprotected barcoded oligonucleotides, for example, by hybridization and/or ligation.

In some aspects, a method disclosed herein provides one or more advantages as compared to other arraying methods. For example, pre-synthesized barcodes can eliminate concerns over barcode fidelity in base-by-base in situ approach. In addition, compared to base-by-base methods, a method disclosed herein can reduce manufacturing production time, cost of goods, and increase total yield. For example, only three or four rounds of hybridization and/or ligation may be required compared to 12-16 rounds of hybridization and/or ligation in a typical base-by-base in situ arraying method. In one aspect, the method disclosed herein does not involve 5′ to 3′ base-by-base synthesis of a polynucleotide in situ on a substrate. In another aspect, there is no need for decoding as all barcodes are synthesized in defined locations on an array and all arrays are identical with respect to each other. In some aspects, feature scaling can readily be increased or decreased by changing photomasks and corresponding barcode diversity. In other aspects, a method disclosed herein is performed on a transparent substrate. Since a method disclosed herein does not depend on the use of microspheres (e.g., barcoded beads) to generate an oligonucleotide array, optical distortion or aberrations caused by microspheres (which may not be transparent) during imaging of the oligonucleotide array and/or a sample (e.g., a tissue section) on the array can be avoided.

In some aspects, provided herein is a method of producing an array of polynucleotides. In some embodiments, an array comprises an arrangement of a plurality of features, e.g., each comprising one or more molecules such as a nucleic acid molecule (e.g., a DNA oligonucleotide), and the arrangement is either irregular or forms a regular pattern. The features and/or molecules on an array may be distributed randomly or in an ordered fashion, e.g. in spots that are arranged in rows and columns. Individual features in the array differ from one another based on their relative spatial locations. In some embodiments, the features and/or molecules are collectively positioned on a substrate.

In some embodiments, the method comprises irradiating an array with light. In some embodiments, the irradiation is selective, for example, where one or more photomasks can be used such that only one or more specific regions of the array are exposed to stimuli (e.g., exposure to light such as UV, and/or exposure to heat induced by laser). In some embodiments, the method comprises irradiating a first polynucleotide immobilized on a substrate with a first light while a second polynucleotide immobilized on the substrate is not irradiated with the first light. For instance, the substrate is exposed to the first light when the second polynucleotide is photomasked while the first polynucleotide is not photomasked. Alternatively, a focused light such as laser may be used to irradiate the first polynucleotide but not the second polynucleotide, even when the second polynucleotide is not masked from the light. For example, the distance (pitch) between features may be selected to prevent the laser from stimulating polynucleotides of an adjacent feature.

In some embodiments, the first polynucleotide comprises a first photo-cleavable moiety that inhibits or blocks hybridization and/or ligation to the first polynucleotide, and the second polynucleotide comprises a second photo-cleavable moiety that inhibits or blocks hybridization and/or ligation to the second polynucleotide. In some embodiments, a photo-cleavable moiety disclosed herein is part of a polynucleotide and inhibits or blocks hybridization to the polynucleotide. In some embodiments, the polynucleotide is prevented from hybridization to a nucleic acid such as a splint. In some embodiments, a photo-cleavable moiety disclosed herein is part of a polynucleotide and inhibits or blocks ligation to either end of the polynucleotide, while hybridization of a nucleic acid to the polynucleotide may or may not be inhibited or blocked. For example, the photo-cleavable moiety may inhibit or block the 3′ or 5′ end of the polynucleotide from chemical or enzymatic ligation, e.g., even when a splint may hybridize to the polynucleotide in order to bring a ligation partner in proximity to the 3′ or 5′ end of the polynucleotide. In some embodiments, the photo-cleavable moiety may cap the 3′ or 5′ end of the polynucleotide.

In some embodiments, the irradiation results in cleavage of the first photo-cleavable moiety such that the inhibition or blocking of hybridization and/or ligation to the first polynucleotide is reduced or eliminated, whereas hybridization and/or ligation to the second polynucleotide remains inhibited or blocked by the second photo-cleavable moiety.

In some embodiments, the method further comprises attaching a first barcode to the first polynucleotide via hybridization and/or ligation. In some embodiments, one end of the barcode and one end of the polynucleotide may be directly ligated, e.g., using a ligase having a single-stranded DNA/RNA ligase activity such as a T4 DNA ligase or CircLigase™. The attachment may comprise hybridizing the first barcode and the first polynucleotide to a splint, wherein one end of the first barcode and one end of the first polynucleotide are in proximity to each other. For example, the 3′ end of the first barcode and the 5′ end of the first polynucleotide may hybridize to a splint. Alternatively, the 5′ end of the first barcode and the 3′ end of the first polynucleotide are in proximity to each other. In some embodiments, proximity ligation is used to ligate a nick, with or without a gap-filling step that involves incorporation of one or more nucleic acids by a polymerase, based on the nucleic acid sequence of the splint which serves as a template.

In some embodiments, the method comprises irradiating a first polynucleotide immobilized on a substrate with a first light while a second polynucleotide immobilized on the substrate is not irradiated with the first light, wherein the first polynucleotide comprises a first photo-cleavable moiety that inhibits or blocks hybridization and/or ligation to the first polynucleotide, and the second polynucleotide comprises a second photo-cleavable moiety that inhibits or blocks hybridization and/or ligation to the second polynucleotide, thereby cleaving the first photo-cleavable moiety such that the inhibition or blocking of hybridization and/or ligation to the first polynucleotide is reduced or eliminated, whereas hybridization and/or ligation to the second polynucleotide remains inhibited or blocked by the second photo-cleavable moiety, and wherein a first barcode is attached to the first polynucleotide via hybridization and/or ligation.

In some embodiments, a first polynucleotide immobilized on a substrate is irradiated with a first light while a second polynucleotide immobilized on the substrate is not irradiated with the first light, wherein the first polynucleotide comprises a first photo-cleavable moiety that inhibits or blocks hybridization and/or ligation to the first polynucleotide, and the second polynucleotide comprises a second photo-cleavable moiety that inhibits or blocks hybridization and/or ligation to the second polynucleotide, and the first photo-cleavable moiety is cleaved such that the inhibition or blocking of hybridization and/or ligation to the first polynucleotide is reduced or eliminated, whereas hybridization and/or ligation to the second polynucleotide remains inhibited or blocked by the second photo-cleavable moiety. In some embodiments, the method comprises attaching a first barcode to the first polynucleotide via hybridization and/or ligation.

In any of the embodiments herein, the method can be used to provide on a substrate an array comprising the first and second polynucleotides, wherein the first polynucleotide is barcoded with the first barcode and the second polynucleotide is not.

In any of the embodiments herein, the method can further comprise irradiating the second polynucleotide with a second light, thereby cleaving the second photo-cleavable moiety such that the inhibition or blocking of hybridization and/or ligation to the second polynucleotide is reduced or eliminated. In any of the embodiments herein, the second polynucleotide can be irradiated with the second light while the first polynucleotide is not irradiated with the second light, for example, due to the use of a photomask that protects the first polynucleotide from light.

In any of the embodiments herein, physical masks, e.g., a photolithography mask which can include an opaque plate or film with transparent areas that allow light to shine through in a defined pattern, may be used.

In any of the embodiments herein, the first light and the second light can be the same, or can be different in at least one attribute, e.g., wavelength, duration and/or intensity, for example, because different protection groups and/or photolabile groups may be used. In any of the embodiments herein, the first light and the second light can have a wavelength between about 365 nm and about 440 nm, for example, about 366 nm, 405 nm, or 436 nm. In some embodiments, the irradiation step herein can be performed for a duration of between about 1 minute and about 10 minutes, for example, for about 2 minutes, about 4 minutes, about 6 minutes, or about 8 minutes. In some embodiments, the irradiation can be performed at a total light dose of between about one and about ten mW/mm², for example, at about 2 mW/mm², about 4 mW/mm², about 6 mW/mm², or about 8 mW/mm². In some embodiments, the irradiation can be performed at a total light dose of between about one and about ten mW/mm² and for a duration of between about 1 minute and about 10 minutes.

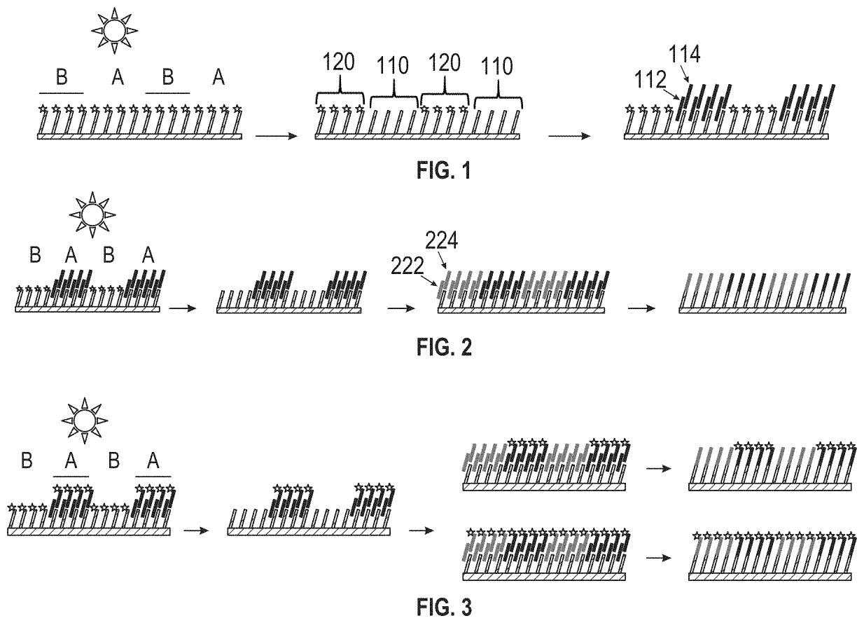

FIG. 1 provides a non-limiting example. A first polynucleotide (e.g., an oligonucleotide) (110) is deposited in a region A of an array and a second polynucleotide (e.g., an oligonucleotide) (120) is deposited in a region B. Regions A are exposed to light while regions B are photomasked. While two regions A and two regions B are shown as adjacent regions, a photomask can be selected and/or adjusted to allow any suitable number and/or combination of regions on the substrate to be exposed to light or masked. Thus, the exposed region(s) and masked region(s) can be in any suitable pattern, which can be predetermined and/or adjusted as needed during the arraying process. In addition, a mirror, mirror array, a lens, a moving stage, and/or a photomask can be used to direct the light to or away from the region(s) of interest. In FIG. 1, the first polynucleotide and the second polynucleotide can comprise the same sequence or different sequences. For example, first polynucleotides (110) in region A and second polynucleotides (120) in region B may form a lawn of universal oligonucleotide molecules on the substrate. The oligonucleotides may be attached to the substrate at their 5′ ends or 3′ ends. The first and second polynucleotides each comprises a first photo-cleavable moiety and a second photo-cleavable moiety (shown as stars), respectively. The first photo-cleavable moieties can be the same or different among the plurality of first polynucleotides. The second photo-cleavable moieties can be the same or different among the plurality of second polynucleotides. In addition, the first and second photo-cleavable moieties in FIG. 1 can be the same or different. While the photo-cleavable moieties are shown at one end of the polynucleotides, they can be attached to any part of the polynucleotides. For example, one or more photo-caged nucleobases may be incorporated throughout the length of a polynucleotide, for example, to render the polynucleotide unavailable for hybridization and/or ligation. In other examples, a photo-cleavable linker connects the polynucleotide to a sequence complementary to a region in the polynucleotide, thereby forming a hairpin or stem-loop structure that renders the polynucleotide unavailable for hybridization and/or ligation. Once regions A are exposed to light to deprotect the first polynucleotide (110) while the second polynucleotide in regions B (120) remain protected, a first barcode can be attached to the first polynucleotide. As an example, FIG. 1 shows a hybridization complex between the first polynucleotide, a splint (112), and a polynucleotide comprising a first barcode (e.g., a round 1 barcode 1A) (114). The polynucleotide comprising the first barcode (114) comprises at least a first barcode sequence and a hybridization region that hybridizes to the splint (112) which is a first splint, and may further comprise a hybridization region that hybridizes to a round 2 splint (e.g., for attaching a round 2 barcode after the round 1 barcode 1A). The first splint (112) comprises at least a hybridization region that hybridizes to the first polynucleotide and a hybridization region that hybridizes to the polynucleotide comprising the first barcode. Optionally, the polynucleotide comprising the first barcode (114) may be ligated to the first polynucleotide, with or without gap filling using the first splint as a template. As a result, provided in FIG. 1 is an array comprising the first and second polynucleotides, wherein the first polynucleotide is barcoded with the first barcode and the second polynucleotide is not.

As shown in FIG. 1, the polynucleotide comprising the first barcode may comprise no photo-cleavable moiety that blocks hybridization and/or ligation. In these examples, the array may be exposed to light to deprotect the second polynucleotide by cleaving the second photo-cleavable moiety, as shown in FIG. 2, and a second barcode can be attached to the second polynucleotide. For example, FIG. 2 shows a hybridization complex between the second polynucleotide, a second splint (222), and a polynucleotide comprising a second barcode (e.g., a round 1 barcode 1B) (224). The polynucleotide comprising the second barcode (224) comprises at least a second barcode sequence and a hybridization region that hybridizes to the second splint (222), and may further comprise a hybridization region that hybridizes to a round 2 splint (e.g., for attaching a round 2 barcode after the round 1 barcode 1). The second splint comprises at least a hybridization region that hybridizes to the second polynucleotide and a hybridization region that hybridizes to the polynucleotide comprising the second barcode. While the polynucleotide comprising the first barcode may be available for hybridization and/or ligation, the second barcode may be specifically attached to the second polynucleotide but not to the first polynucleotide barcoded with the first barcode. For example, the sequence of the second splint may be selected such that it specifically hybridizes to the second polynucleotide but not to the polynucleotide comprising the first barcode. In these examples, both the first barcode (e.g., barcode 1A) and the second barcode (e.g., barcode 1B) are round 1 barcodes.

Optionally, the polynucleotides comprising the first/second barcodes may be ligated to the first/second polynucleotides, respectively, with or without gap filling using the first/second splints as templates. As a result, provided in FIG. 2 is an array comprising the first and second polynucleotides barcoded with the first barcode and the second barcode, respectively. Neither of the barcoded polynucleotides comprises a photo-cleavable moiety.

Alternatively, the polynucleotide comprising the first barcode may comprise a photo-cleavable moiety that blocks hybridization and/or ligation, for example as shown in FIG. 3. The photo-cleavable moiety may be the same as the first photo-cleavable moiety (of the first polynucleotide) and/or the second photo-cleavable moiety (of the second polynucleotide). In other embodiments, the photo-cleavable moiety is different from either or both of the first photo-cleavable moiety (of the first polynucleotide) and the second photo-cleavable moiety (of the second polynucleotide). In these examples, regions B may be exposed to light to deprotect the second polynucleotide by cleaving the second photo-cleavable moiety, while regions A containing the first polynucleotide barcoded with the first barcode are photomasked. After light exposure and removal of the photomask, the array comprises the deprotected second polynucleotide, which is available for hybridization and/or ligation, and the first polynucleotide protected by the photo-cleavable moiety of the polynucleotide comprising the first barcode, which is not available for hybridization and/or ligation.

A second barcode can be attached to the second polynucleotide, as shown in FIG. 3. For example, the polynucleotide comprising the second barcode may comprise no photo-cleavable moiety that blocks hybridization and/or ligation, for example, as shown in FIG. 3 (upper branch). In these examples, regions A contain the first polynucleotide barcoded with the first barcode which comprises a photo-cleavable moiety, while regions B contain the second polynucleotide barcoded with the second barcode which comprises no photo-cleavable moiety. Optionally, the polynucleotides comprising the first/second barcodes may be ligated to the first/second polynucleotides, respectively, with or without gap filling using the first/second splints as templates. As a result, provided in FIG. 3 (upper branch) is an array comprising the first and second polynucleotides barcoded with the first barcode and the second barcode, respectively. While the first polynucleotide barcoded with the first barcode (in regions A) comprises a photo-cleavable moiety, the second polynucleotide barcoded with the second barcode (in regions B) does not. In some examples, polynucleotides in regions A may undergo one or more additional rounds of barcoding. While the polynucleotides in regions B may be available for hybridization and/or ligation, the round 2 barcode may be specifically attached to the polynucleotides in regions A but not to those in regions B. For example, the sequence of the round 2 splint may be selected such that it specifically hybridizes to the polynucleotide comprising the first barcode (a round 1 barcode) but not to the polynucleotide comprising the second barcode (also a round 1 barcode). Thus, the round 2 barcode is attached to polynucleotides in regions A which have received a round 1 barcode.

Alternatively, the polynucleotide comprising the second barcode may comprise a photo-cleavable moiety that blocks hybridization and/or ligation, for example, as shown in FIG. 3 (lower branch). The photo-cleavable moiety may be the same as the first photo-cleavable moiety (of the first polynucleotide), the second photo-cleavable moiety (of the second polynucleotide), and/or the photo-cleavable moiety of the polynucleotide comprising the first barcode. In other embodiments, the photo-cleavable moiety is different from any one or more of the first photo-cleavable moiety (of the first polynucleotide), the second photo-cleavable moiety (of the second polynucleotide), and the photo-cleavable moiety of the polynucleotide comprising the first barcode. In these examples, regions A contain the first polynucleotide barcoded with the first barcode which comprises a photo-cleavable moiety, while regions B contain the second polynucleotide barcoded with the second barcode which also comprises a photo-cleavable moiety. Optionally, the polynucleotides comprising the first/second barcodes may be ligated to the first/second polynucleotides, respectively, with or without gap filling using the first/second splints as templates. As a result, provided in FIG. 3 (lower branch) is an array comprising the first and second polynucleotides barcoded with the first barcode and the second barcode, respectively, and neither of the barcoded polynucleotides is available for hybridization and/or ligation. The splint molecules may be optionally removed.

In some examples, polynucleotides in regions A and/or polynucleotides in regions B may undergo one or more additional rounds of barcoding. For example, after the round 1 barcoding, regions A may contain polynucleotides P1 and P3 each barcoded with round 1 barcode 1A (i.e., polynucleotides 1A-P1 and 1A-P3) and regions B may contain polynucleotides P2 and P4 each barcoded with round 1 barcode 1B (i.e., polynucleotides 1B-P2 and 1B-P4). All of polynucleotides 1A-P1, 1A-P3, 1B-P2, and 1B-P4 may be protected by a photo-cleavable moiety, for example, as shown in FIG. 3 (lower branch). With light exposure and photomasking, any one or more of polynucleotides 1A-P1 and 1A-P3 (in regions A) and 1B-P2 and 1B-P4 (in regions B) may undergo a second round of barcoding.

For instance, a round 2 barcode 2A may be attached to any one of polynucleotides 1A-P1, 1A-P3, 1B-P2, and 1B-P4. In some embodiments, a round 2 barcode 2A may be attached to any two of polynucleotides 1A-P1, 1A-P3, 1B-P2, and 1B-P4. In some embodiments, a round 2 barcode 2A may be attached to any three of polynucleotides 1A-P1, 1A-P3, 1B-P2, and 1B-P4. In some embodiments, a round 2 barcode 2A may be attached to all of polynucleotides 1A-P1, 1A-P3, 1B-P2, and 1B-P4.

In other examples, different round 2 barcodes 2A and 2B may be used. In some embodiments, barcode 2A is attached to polynucleotides 1A-P1 and 1A-P3 (in regions A) while barcode 2B is attached to polynucleotides 1B-P2 and 1B-P4 (in regions B). For higher order rounds, for example, round m (m being an integer of 2 or greater), the regions A polynucleotides may receive barcode mA while the regions B polynucleotides receive barcode mB. Barcodes mA and mB may be the same or different in sequence. Thus, for each round, the regions A polynucleotides (e.g., P1 and P3) and the regions B polynucleotides (e.g., P2 and P4) may have no crossover, generating barcoded polynucleotides mA- . . . -1A-P1 and mA- . . . -1A-P3 (in regions A) and mB- . . . -1B-P2 and mB- . . . -1B-P4 (in regions B).

Alternatively, the regions A polynucleotides (e.g., P1 and P3) and the regions B polynucleotides (e.g., P2 and P4) may have crossover. For example, barcode 2A is attached to polynucleotides 1A-P1 (in regions A) and 1B-P2 (in regions B) while barcode 2B is attached to polynucleotides 1A-P3 (in regions A) and 1B-P4 (in regions B). For round m (m being an integer of 2 or greater), one or more of the regions A polynucleotides and/or one or more of the regions B polynucleotides may receive barcode mA, while one or more of the regions A polynucleotides and/or one or more of the regions B polynucleotides barcode mB. Barcodes mA and mB may be the same or different in sequence.

In some examples, round m (m being an integer of 2 or greater) barcodes mA, mB, and mC may be attached to any polynucleotides barcoded in the previous round (i.e., round m−1), and mA, mB, and mC may be the same or different. In other examples, round m (m being an integer of 2 or greater) barcodes mA, mB, mC, and mD may be attached to any polynucleotides barcoded in the previous round (i.e., round m−1), and mA, mB, mC, and mD may be the same or different.

In any of the embodiments herein, the barcoding rounds can be repeated m times to achieve a desired barcode diversity, m being an integer of 2 or greater. In some embodiments, m is 3, 4, 5, 6, 7, 8, 9, or 10, or greater than 10. In any of the embodiments herein, each of the m barcoding rounds may comprise n cycles (each cycle for molecules in one or more features), wherein integer n is 2 or greater and independent of m. In some embodiments, n is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or greater than 50.

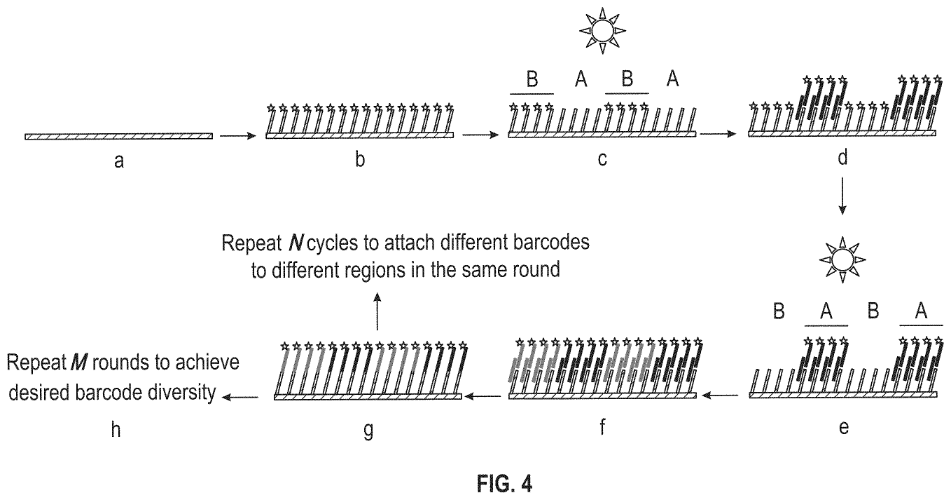

FIG. 4 provides another non-limiting example. In FIG. 4a, a substrate is provided. The substrate comprises a surface for nucleic acids to be deposited on and can be in the form of a slide, such as a glass slide or a wafer, such a silicon dioxide wafer. In some examples, the substrate is transparent. In FIG. 4b, a lawn of polynucleotides comprising photo-cleavable moieties, such as photo-caged oligos, are deposited on the substrate and immobilized. In FIG. 4c, one or more regions (e.g., regions A) on the substrate are exposed to light in order to cleave the photo-cleavable moieties and uncage the polynucleotides, rendering them available for hybridization and/or ligation, while one or more other regions (e.g., regions B) on the substrate are masked, for example, using a photomask such as those in photolithography. Patterned access to the uncaged polynucleotides on the underlying substrate is provided, and in FIG. 4d, a round 1 barcode (such as barcode 1A) may be attached to the uncaged polynucleotides via hybridization and/or ligation. For example, an oligo may be used to hybridize to an uncaged polynucleotide and a polynucleotide comprising the round 1 barcode. In some examples, barcodes 1A are attached via hybridization to the oligonucleotide and are not ligated to the polynucleotides immobilized on the substrate in regions A. Alternatively, the oligonucleotide may comprise a splint that facilitates proximity ligation of one end of the uncaged polynucleotide and one end of the polynucleotide comprising the round 1 barcode, thus attaching the barcode to the uncaged polynucleotide. The proximity ligation may occur immediately following FIG. 4d or in a subsequent step, e.g., following FIG. 4f as described below. The polynucleotide comprising the round 1 barcode may further comprise a photo-cleavable moiety that inhibits or blocks hybridization and/or ligation to the uncaged polynucleotide barcoded with the round 1 barcode. Thus, after barcode attachment, the uncaged polynucleotide becomes caged, and a lawn of photo-caged oligos are again provided on the substrate, albeit some are barcoded while others are not. In FIG. 4e, one or more regions on the substrate (e.g., regions B) are exposed to light in order to cleave the photo-cleavable moieties and uncage the polynucleotides, rendering them available for hybridization and/or ligation, while one or more other regions on the substrate are photomasked. For instance, the polynucleotides barcoded with 1A in regions A are masked while regions B are exposed to light in order to cleave the photo-cleavable moieties and uncage the polynucleotides in regions B. In FIG. 4f, another round 1 barcode (such as barcode 1B) may be attached to the uncaged polynucleotides in regions B via hybridization and/or ligation. For example, an oligonucleotide may be used to hybridize to an uncaged polynucleotide in regions B and a polynucleotide comprising barcode 1B. Optionally, the oligonucleotide may comprise a splint that facilitates proximity ligation of one end of the uncaged polynucleotide and one end of the polynucleotide comprising the round 1 barcode, thus attaching the barcode to the uncaged polynucleotide. The polynucleotide comprising barcode 1B may further comprise a photo-cleavable moiety that inhibits or blocks hybridization and/or ligation. Thus, after barcode 1B attachment, a lawn of photo-caged oligonucleotides are again provided on the substrate, albeit some are barcoded with barcode 1A while others are barcoded with barcode 1B. In the example shown in FIG. 4f, barcodes 1A and 1B are attached via hybridization to splints and are not ligated to the polynucleotides immobilized on the substrate. Optional proximity ligation and/or removal of the splints may be performed to provide a lawn of photo-caged single-stranded oligonucleotides in FIG. 4g. Processes similar to the round 1 barcoding steps may be repeated to achieve a desired barcode diversity in FIG. 4h, and a different photomasking pattern may be used in each barcoding round. The irradiation-hybridization-ligation steps can be repeated for N cycles, each cycle for one or more different pre-determined regions (e.g., features) on the substrate. After all regions (e.g., features) are ligated to barcodes of a particular round, the cycles may be repeated in one or more rounds. In some cases, the round is repeated M times to ligate M parts of a barcode onto the substrate, generating a nucleotide array with N^{M} sequence diversity.

In one aspect, provided herein is a method for providing differentially barcoded polynucleotides, comprising: (i) irradiating a first region on a substrate with a first light while a second region on said substrate is not irradiated with said first light, wherein: said first region comprises a first plurality of polynucleotides immobilized on said substrate and said second region comprises a second plurality of polynucleotides immobilized on said substrate, said first region and said second region each comprises a first photo-cleavable agent that inhibits or blocks hybridization and/or ligation to said first plurality of polynucleotides and said second plurality of polynucleotides, respectively, whereby said irradiation cleaves said first photo-cleavable agent such that said inhibition or blocking of hybridization and/or ligation to said first plurality of polynucleotides is reduced or eliminated, whereas hybridization and/or ligation to said second plurality of polynucleotides remains inhibited or blocked by said first photo-cleavable agent; (ii) attaching a first barcode to said first plurality of polynucleotides via hybridization and/or ligation; (iii) irradiating said second region with a second light while said first region is not irradiated with said second light, thereby cleaving said first photo-cleavable agent such that said inhibition or blocking of hybridization and/or ligation to said second plurality of polynucleotides is reduced or eliminated; (iv) attaching a second barcode to said second plurality of polynucleotides via hybridization and/or ligation, wherein said second barcode sequence is different from said first barcode sequence, thereby providing differentially barcoded polynucleotides on said substrate.

In another aspect, provided herein is a method for providing differentially barcoded polynucleotides, comprising: (i) irradiating a first region on a substrate with a first light while a second region on said substrate is not irradiated with said first light, wherein: said first region comprises a first plurality of polynucleotides immobilized on said substrate and said second region comprises a second plurality of polynucleotides immobilized on said substrate, said first plurality of polynucleotides and said second plurality of polynucleotides each comprise a photo-cleavable moiety that inhibits or blocks hybridization and/or ligation to said first plurality of polynucleotides and said second plurality of polynucleotides, respectively, whereby said irradiation cleaves said photo-cleavable moiety such that said inhibition or blocking of hybridization and/or ligation to said first plurality of polynucleotides is reduced or eliminated, whereas hybridization and/or ligation to said second plurality of polynucleotides remains inhibited or blocked by said photo-cleavable moiety; (ii) attaching a first barcode to said first plurality of polynucleotides via hybridization and/or ligation; (iii) irradiating said second region with a second light while said first region is not irradiated with said second light, thereby cleaving said photo-cleavable moiety such that said inhibition or blocking of hybridization and/or ligation to said second plurality of polynucleotides is reduced or eliminated; (iv) attaching a second barcode to said second plurality of polynucleotides via hybridization and/or ligation, wherein said second barcode sequence is different from said first barcode sequence, thereby providing differentially barcoded polynucleotides on said substrate.

In another aspect, provided herein is a method for providing differentially barcoded polynucleotides, comprising: (i) irradiating a first region on a substrate with a first light while a second region on said substrate is not irradiated with said first light, wherein: said first region comprises a first plurality of polynucleotides immobilized on said substrate and said second region comprises a second plurality of polynucleotides immobilized on said substrate, said first plurality of polynucleotides and said second plurality of polynucleotides each comprise a first photo-cleavable moiety that inhibits or blocks hybridization and/or ligation to said first plurality of polynucleotides and said second plurality of polynucleotides, respectively, whereby said irradiation cleaves said first photo-cleavable moiety such that said inhibition or blocking of hybridization and/or ligation to said first plurality of polynucleotides is reduced or eliminated, whereas hybridization and/or ligation to said second plurality of polynucleotides remains inhibited or blocked by said first photo-cleavable moiety; (ii) attaching a first barcode to said first plurality of polynucleotides via hybridization and/or ligation, wherein each first barcode comprises a second photo-cleavable moiety that inhibits or blocks hybridization and/or ligation to said first barcode; (iii) irradiating said second region with a second light while said first region is not irradiated with said second light, thereby cleaving said first photo-cleavable moiety such that said inhibition or blocking of hybridization and/or ligation to said second plurality of polynucleotides is reduced or eliminated, whereas hybridization and/or ligation to each first barcode remain inhibited or blocked by said second photo-cleavable moiety; (iv) attaching a second barcode to said second plurality of polynucleotides via hybridization and/or ligation, wherein said second barcode sequence is different from said first barcode sequence, and wherein each second barcode comprises a third photo-cleavable moiety that inhibits or blocks hybridization and/or ligation to said second barcode, thereby providing differentially barcoded polynucleotides on said substrate.

In another aspect, provided herein is a method for providing differentially barcoded polynucleotides, comprising: (i) irradiating a first region on a substrate with a first light while a second region on said substrate is not irradiated with said first light, wherein: said first region comprises a first plurality of polynucleotides immobilized on said substrate and said second region comprises a second plurality of polynucleotides immobilized on said substrate, said first plurality of polynucleotides and said second plurality of polynucleotides each comprise a first photo-cleavable moiety that inhibits or blocks ligation to said first plurality of polynucleotides and said second plurality of polynucleotides, respectively, whereby said irradiation cleaves said first photo-cleavable moiety such that said inhibition or blocking of ligation to said first plurality of polynucleotides is reduced or eliminated, whereas ligation to said second plurality of polynucleotides remains inhibited or blocked by said first photo-cleavable moiety; (ii) attaching a first barcode to said first plurality of polynucleotides via ligation, wherein each first barcode comprises a second photo-cleavable moiety that inhibits or blocks ligation to said first barcode; (iii) irradiating said second region with a second light while said first region is not irradiated with said second light, thereby cleaving said first photo-cleavable moiety such that said inhibition or blocking of ligation to said second plurality of polynucleotides is reduced or eliminated, whereas ligation to each first barcode remains inhibited or blocked by said second photo-cleavable moiety; (iv) attaching a second barcode to said second plurality of polynucleotides via ligation, wherein said second barcode sequence is different from said first barcode sequence, and wherein each second barcode comprises a third photo-cleavable moiety that inhibits or blocks ligation to said second barcode, thereby providing differentially barcoded polynucleotides on said substrate.

In another aspect, provided herein is a method for providing differentially barcoded polynucleotides, comprising: (i) irradiating a first region on a substrate with a first light while a second region on said substrate is not irradiated with said first light, wherein: said first region comprises a first plurality of polynucleotides immobilized on said substrate and said second region comprises a second plurality of polynucleotides immobilized on said substrate, said first plurality of polynucleotides and said second plurality of polynucleotides each comprise a first photo-cleavable moiety that inhibits or blocks ligation to said first plurality of polynucleotides and said second plurality of polynucleotides, respectively, whereby said irradiation cleaves said first photo-cleavable moiety such that said inhibition or blocking of ligation to said first plurality of polynucleotides is reduced or eliminated, whereas ligation to said second plurality of polynucleotides remain inhibited or blocked by said first photo-cleavable moiety; (ii) attaching a first barcode to said first plurality of polynucleotides via hybridization to a first splint followed by ligation of said first barcode to said first plurality of polynucleotides, wherein each first barcode comprises a first hybridization region that hybridizes to said first splint, a first barcode region, and a second photo-cleavable moiety that inhibits or blocks ligation to said first barcode; (iii) irradiating said second region with a second light while said first region is not irradiated with said second light, thereby cleaving said first photo-cleavable moiety such that said inhibition or blocking of ligation to said second plurality of polynucleotides is reduced or eliminated, whereas ligation to each first barcode remains inhibited or blocked by said second photo-cleavable moiety; (iv) attaching a second barcode to said second plurality of polynucleotides via hybridization to a second splint followed by ligation of said second barcode to said second plurality of polynucleotides, wherein each second barcode comprises a second hybridization region that hybridizes to said second splint, a second barcode region, and a third photo-cleavable moiety that inhibits or blocks ligation to said second barcode, and wherein said second barcode region sequence is different from said first barcode region sequence, thereby providing differentially barcoded polynucleotides on said substrate.

In some embodiments, the first polynucleotide (e.g., in regions A) and the second polynucleotide (e.g., in regions B) initially deposited on the substrate are of the same nucleic acid sequence. In other embodiments, the first polynucleotide (e.g., in regions A) and the second polynucleotide (e.g., in regions B) initially deposited on the substrate are of different nucleic acid sequences, and the substrate may be pre-patterned. In some embodiments, prior to the light-controlled surface patterning in situ, barcodes have been attached to a lawn of universal oligonucleotides on the substrate, e.g., in a known pattern.

In some embodiments, provided herein is a method for providing an array of polynucleotides, comprising: (a1) irradiating polynucleotide P1 immobilized on a substrate with light while polynucleotide P2 immobilized on the substrate is photomasked, wherein polynucleotides P1 and P2 comprise a photo-cleavable moiety that inhibits or blocks hybridization and/or ligation to P1 and P2, respectively, thereby cleaving the photo-cleavable moiety to allow hybridization and/or ligation to P1, whereas hybridization and/or ligation to P2 remains inhibited or blocked by the photo-cleavable moiety; and (b1) attaching barcode 1A to P1 via hybridization and/or ligation to form a barcoded polynucleotide 1A-P1, wherein barcode 1A comprises the photo-cleavable moiety which inhibits or blocks hybridization and/or ligation to 1A-P1, thereby providing on the substrate an array comprising polynucleotides 1A-P1 and P2 each comprising the photo-cleavable moiety that inhibits or blocks hybridization and/or ligation.

In some embodiments, the method further comprises: (c1) irradiating P2 with light while 1A-P1 is photomasked, thereby cleaving the photo-cleavable moiety to allow hybridization and/or ligation to P2, whereas hybridization and/or ligation to 1A-P1 remains inhibited or blocked by the photo-cleavable moiety; and (d1) attaching barcode 1B to P2 via hybridization and/or ligation to form a barcoded polynucleotide 1B-P2, wherein barcode 1B comprises the photo-cleavable moiety which inhibits or blocks hybridization and/or ligation to 1B-P2, thereby providing on the substrate an array comprising barcoded polynucleotides 1A-P1 and 1B-P2 each comprising the photo-cleavable moiety that inhibits or blocks hybridization and/or ligation.

In some embodiments, barcodes 1A and 1B comprise the same nucleic acid sequence. In other embodiments, barcodes 1A and 1B comprise different nucleic acid sequences.

In some embodiments, the method further comprises: (a2) irradiating one of 1A-P1 and 1B-P2 with light while the other is photomasked, thereby cleaving the photo-cleavable moiety to allow hybridization and/or ligation to the irradiated polynucleotide, whereas hybridization and/or ligation to the photomasked polynucleotide remains inhibited or blocked by the photo-cleavable moiety; and (b2) attaching barcode 2A to the irradiated polynucleotide via hybridization and/or ligation to form a 2A-barcoded polynucleotide, wherein barcode 2A comprises the photo-cleavable moiety which inhibits or blocks hybridization and/or ligation, thereby providing on the substrate an array comprising barcoded polynucleotides each comprising the photo-cleavable moiety that inhibits or blocks hybridization and/or ligation.

In some embodiments, the method further comprises: (c2) irradiating the photomasked polynucleotide in step a2 with light while the 2A-barcoded polynucleotide is photomasked, thereby cleaving the photo-cleavable moiety to allow hybridization and/or ligation, whereas hybridization and/or ligation to the 2A-barcoded polynucleotide remains inhibited or blocked by the photo-cleavable moiety; and (d2) attaching barcode 2B to the irradiated polynucleotide in step c2 via hybridization and/or ligation to form a 2B-barcoded polynucleotide, wherein barcode 2B comprises the photo-cleavable moiety which inhibits or blocks hybridization and/or ligation, thereby providing on the substrate an array comprising barcoded polynucleotides each comprising the photo-cleavable moiety that inhibits or blocks hybridization and/or ligation.

In some embodiments, the barcoded polynucleotides on the array comprise polynucleotides 2A-1A-P1 and 2B-1B-P2. In other embodiments, the barcoded polynucleotides on the array comprise polynucleotides 2B-1A-P1 and 2A-1B-P2.

In some embodiments, steps a1-d1 form round 1 and steps a2-d2 form round 2, and the method further comprises steps ai-di in round i, wherein barcodes iA and iB are attached to provide barcoded polynucleotides on the substrate, and wherein i is an integer greater than 2. In some embodiments, barcodes iA and iB comprise the same nucleic acid sequence. In other embodiments, barcodes iA and iB comprise different nucleic acid sequences.

In some embodiments, provided herein is a method for providing an array of polynucleotides, comprising: (a) irradiating polynucleotide P1 and polynucleotide P3 immobilized on a substrate with light while polynucleotide P2 and polynucleotide P4 immobilized on the substrate are photomasked, wherein polynucleotides P1-P4 comprise a photo-cleavable moiety that inhibits or blocks hybridization and/or ligation to P1-P4, respectively, thereby cleaving the photo-cleavable moiety to allow hybridization and/or ligation to P1 and P3, whereas hybridization and/or ligation to P2 and P4 remain inhibited or blocked by the photo-cleavable moiety; (b) attaching barcode 1A to P1 and P3 via hybridization and/or ligation to form barcoded polynucleotides 1A-P1 and 1A-P3, wherein barcode 1A comprises the photo-cleavable moiety which inhibits or blocks hybridization and/or ligation; (c) irradiating P2 and P4 with light while 1A-P1 and 1A-P3 are photomasked, thereby cleaving the photo-cleavable moiety to allow hybridization and/or ligation to P2 and P4, whereas hybridization and/or ligation to 1A-P1 and 1A-P3 remains inhibited or blocked by the photo-cleavable moiety; and (d) attaching barcode 1B to P2 and P4 via hybridization and/or ligation to form barcoded polynucleotides 1B-P2 and 1B-P4, wherein barcode 1B comprises the photo-cleavable moiety which inhibits or blocks hybridization and/or ligation, thereby providing on the substrate an array comprising barcoded polynucleotides 1A-P1, 1B-P2, 1A-P3, and 1B-P4, each comprising the photo-cleavable moiety that inhibits or blocks hybridization and/or ligation.

In some embodiments, the method further comprises (a′) irradiating polynucleotides 1A-P1 and 1B-P2 with light while polynucleotides 1A-P3 and 1B-P4 are photomasked, thereby cleaving the photo-cleavable moiety to allow hybridization and/or ligation to 1A-P1 and 1B-P2, whereas hybridization and/or ligation to 1A-P3 and 1B-P4 remain inhibited or blocked by the photo-cleavable moiety, (b′) attaching barcode 2A to 1A-P1 and 1B-P2 via hybridization and/or ligation to form barcoded polynucleotides 2A-1A-P1 and 2A-1B-P2, wherein barcode 2A comprises the photo-cleavable moiety which inhibits or blocks hybridization and/or ligation; (c′) irradiating 1A-P3 and 1B-P4 with light while 2A-1A-P1 and 2A-1B-P2 are photomasked, thereby cleaving the photo-cleavable moiety to allow hybridization and/or ligation to 1A-P3 and 1B-P4, whereas hybridization and/or ligation to 2A-1A-P1 and 2A-1B-P2 remain inhibited or blocked by the photo-cleavable moiety; and (d′) attaching barcode 2B to 1A-P3 and 1B-P4 via hybridization and/or ligation to form barcoded polynucleotides 2B-1A-P3 and 2B-1B-P4, wherein barcode 2B comprises the photo-cleavable moiety which inhibits or blocks hybridization and/or ligation, thereby providing on the substrate an array comprising barcoded polynucleotides 2A-1A-P1, 2A-1B-P2, 2B-1A-P3, and 2B-1B-P4, each comprising the photo-cleavable moiety that inhibits or blocks hybridization and/or ligation.

In some embodiments, a photo-cleavable moiety disclosed herein inhibits or blocks hybridization. In some embodiments, the photo-cleavable moiety comprises a photo-caged nucleobase, for example, a photo-caged dA, dT, dC, or dG. In some embodiments, the photo-cleavable moiety comprises a photocleavable linker. In some embodiments, the photo-cleavable linker comprises a nitrobenzyl, nitropiperonyl or anthrylmethyl linker. Any suitable photo-cleavable moiety can be used. For example, suitable photo-cleavable moieties are described in Klan et al., Chem. Rev., (2013), 113(1), 119-91; Liu and Deiters, Acc. Chem. Res., (2014) 47(1), 45-55; and Ikeda and Kabumoto, Chem. Letters, (2017), 46(5), 634-640 and are incorporated herein by reference in their entirety. In some embodiments, the photo-cleavable moiety comprises the following structure:

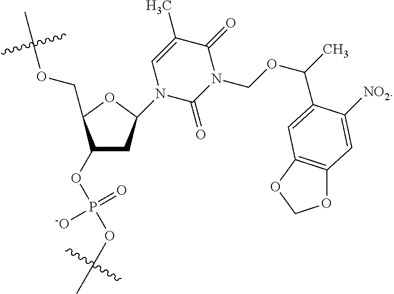

In some embodiments, the photo-cleavable moiety comprises a photo-cleavable hairpin. In some embodiments, the photo-cleavable moiety comprises the following structure:

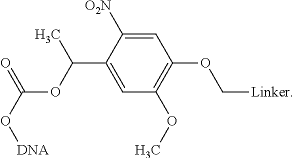

In some embodiments, the photo-cleavable moiety comprises a photo-caged 3-hydroxyl group. In some embodiments, the photo-cleavable moiety comprises the following structure:

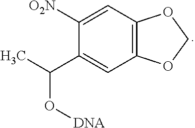

In some embodiments, the first photo-cleavable moiety and/or the second photo-cleavable moiety can comprise a photo-cleavable spacer, for example, a spacer that lacks a 5′ phosphate for ligation. In some embodiments, the photo-cleavable moiety comprises the following structure:

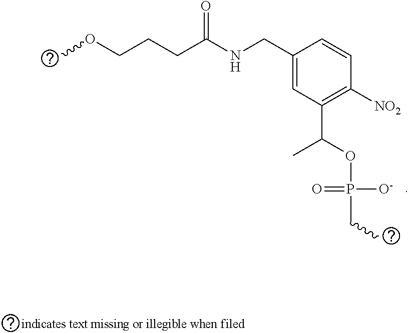

In some embodiments, the photo-cleavable moiety comprises a photo-cleavable polymer.

In another aspect, disclosed herein is a method for providing an array of polynucleotides, comprising: (a) irradiating a plurality of first polynucleotides immobilized on a substrate with a first light while a plurality of second polynucleotides immobilized on the substrate are not irradiated with the first light, wherein each first polynucleotide comprises a first photo-cleavable moiety that inhibits or blocks hybridization and/or ligation to the first polynucleotide, and each second polynucleotide comprises a second photo-cleavable moiety that inhibits or blocks hybridization and/or ligation to the second polynucleotide, thereby cleaving the first photo-cleavable moieties such that the inhibition or blocking of hybridization and/or ligation to the plurality of first polynucleotides is reduced or eliminated, whereas hybridization and/or ligation to the second plurality of polynucleotides remain inhibited or blocked by the second photo-cleavable moieties, and (b) attaching first barcodes to the plurality of first polynucleotide via hybridization and/or ligation, thereby providing on the substrate an array comprising the first polynucleotides barcoded with the first barcodes and the second polynucleotides not barcoded with the first barcodes.

In some embodiments, at least about 90%, at least about 95%, at least about 99%, or 100% of the first barcodes comprise a third photo-cleavable moiety that inhibits or blocks hybridization and/or ligation to the first barcodes.

In some embodiments, at least about 90%, at least about 95%, at least about 99%, or 100% of the first polynucleotides barcoded with the first barcodes comprise the third photo-cleavable moiety.

In some embodiments, the method further comprises: (c) irradiating the plurality of second polynucleotides with a second light, thereby cleaving the second photo-cleavable moieties such that the inhibition or blocking of hybridization and/or ligation to the plurality of second polynucleotides is reduced or eliminated.

In some embodiments, the plurality of second polynucleotides are irradiated with the second light while the plurality of first polynucleotides are not irradiated with the second light.

In some embodiments, the method further comprises: (d) attaching second barcodes to the plurality of second polynucleotides via hybridization and/or ligation, wherein hybridization and/or ligation to the first polynucleotide barcoded with the first barcodes is inhibited or blocked, thereby providing on the substrate an array comprising the first polynucleotides barcoded with the first barcodes and the second polynucleotides barcoded with the second barcodes.

In some embodiments, at least about 90%, at least about 95%, at least about 99%, or 100% of the second barcodes comprise a fourth photo-cleavable moiety that inhibits or blocks hybridization and/or ligation to the second barcodes. In some embodiments, at least about 90%, at least about 95%, at least about 99%, or 100% of the second polynucleotides barcoded with the second barcodes comprise the fourth photo-cleavable moiety. In some embodiments, at least about 90%, at least about 95%, at least about 99%, or 100% of the plurality of first polynucleotides have the same nucleic acid sequences. In some embodiments, at least about 90%, at least about 95%, at least about 99%, or 100% of the plurality of second polynucleotides have the same nucleic acid sequences. In some embodiments, at least about 90%, at least about 95%, at least about 99%, or 100% of the plurality of first polynucleotides have the same nucleic acid sequences as at least about 90%, at least about 95%, at least about 99%, or 100% of the plurality of second polynucleotides.

In some embodiments, the plurality of first polynucleotides and the plurality of second polynucleotides have the same nucleic acid sequences. In some embodiments, polynucleotides of a universal nucleic acid sequence are immobilized on the substrate prior to the irradiation.

In some embodiments, polynucleotides of different nucleic acid sequences are immobilized on the substrate in a pattern prior to the irradiation. In some embodiments, the pattern comprises rows and/or columns. In some embodiments, the pattern comprises regular and/or irregular shapes (e.g., polygons).

In some embodiments, at least about 90%, at least about 95%, at least about 99%, or 100% of the plurality of first polynucleotides are barcoded with the first barcodes, and/or at least about 90%, at least about 95%, at least about 99%, or 100% of the plurality of second polynucleotides are barcoded with the second barcodes.

In yet another aspect, provided herein is a method for providing an array of polynucleotides, comprising: (i) irradiating a first polynucleotide immobilized on a substrate with light while a second polynucleotide immobilized on the substrate is not irradiated with light, wherein the first and second polynucleotides each comprises a photo-cleavable moiety that inhibits or blocks hybridization, thereby cleaving the photo-cleavable moiety to allow hybridization to the first polynucleotide, whereas hybridization to the second polynucleotide remains inhibited or blocked by the photo-cleavable moiety; (ii) attaching a first barcode to the first polynucleotide via hybridization to a first splint followed by ligation, wherein the first splint hybridizes to one end of the first polynucleotide and one end of the first barcode, and wherein the first barcode comprises the photo-cleavable moiety; (iii) irradiating the second polynucleotide with light, thereby cleaving the photo-cleavable moiety to allow hybridization to the second polynucleotide; and (iv) attaching a second barcode to the second polynucleotide via hybridization to a second splint followed by ligation, wherein the second splint hybridizes to one end of the second polynucleotide and one end of the second barcode, and wherein the second barcode comprises the photo-cleavable moiety, thereby providing on the substrate an array comprising the first polynucleotide barcoded with the first barcode and the second polynucleotide barcoded with the second barcode.

In another aspect, provided herein is a method for providing an array of polynucleotides, comprising: (i) irradiating a first polynucleotide immobilized on a substrate with light while a second polynucleotide immobilized on the substrate is not irradiated with light, wherein the first and second polynucleotides each comprises a photo-cleavable moiety that inhibits or blocks ligation to the '3 end of the first and second polynucleotides, respectively, thereby cleaving the photo-cleavable moiety to allow ligation to the '3 end of the first polynucleotide, whereas ligation to the '3 end of the second polynucleotide remains inhibited or blocked by the photo-cleavable moiety; (ii) attaching a first barcode to the first polynucleotide via hybridization to a first splint followed by ligation, wherein the first splint hybridizes to a '3 end portion of the first polynucleotide and a '5 end portion of the first barcode, and wherein the first barcode comprises the photo-cleavable moiety which blocks ligation to the '3 end of the first barcode; (iii) irradiating the second polynucleotide with light, thereby cleaving the photo-cleavable moiety to allow hybridization to the '3 end of the second polynucleotide; and (iv) attaching a second barcode to the second polynucleotide via hybridization to a second splint followed by ligation, wherein the second splint hybridizes to a '3 end portion of the second polynucleotide and a '5 end portion of the second barcode, and wherein the second barcode comprises the photo-cleavable moiety which blocks ligation to the '3 end of the second barcode, thereby providing on the substrate an array comprising the first polynucleotide barcoded with the first barcode and the second polynucleotide barcoded with the second barcode.

In any of the embodiments herein, pre-patterning the substrate may be used prior to the light-controlled surface patterning in situ. For instance, when an initial layer of oligonucleotides on a surface is pre-patterned, the number of cycles and/or rounds of photo-uncaging, hybridization, and ligation may be reduced. In some embodiments, positive photoresist exposure and developing are used to create a patterned surface to allow immobilization of oligonucleotides only at specified surface locations, for examples, in rows and/or columns. Suitable photoresists have been described, for example, in U.S. Patent Pub. No. 20200384436 and U.S. Patent Pub. No. 20210017127, the content of which is herein incorporated by reference in its entirety. In some embodiments, the patterned surface may comprise wells, and each well receives a unique oligonucleotide, e.g., one having a sequencing adapter (e.g., partial or complete Read1)-Unique molecular identifier (UMI)-barcode1-bridge sequence (splint). The bridge sequence may or may not comprise a photo-cleavable protective group.

In some embodiments, the molecules on an array comprise oligonucleotide barcodes. A barcode sequence can be of varied length. In some embodiments, the barcode sequence is about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, or about 70 nucleotides in length. In some embodiments, the barcode sequence is between about 4 and about 25 nucleotides in length. In some embodiments, the barcode sequences is between about 10 and about 50 nucleotides in length. The nucleotides can be completely contiguous, i.e., in a single stretch of adjacent nucleotides, or they can be separated into two or more separate subsequences that are separated by 1 or more nucleotides. In some embodiments, the barcode sequence can be about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or longer. In some embodiments, the barcode sequence can be at least about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or longer. In some embodiments, the barcode sequence can be at most about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or shorter.

The oligonucleotide can include one or more (e.g., two or more, three or more, four or more, five or more) Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain).

A UMI can be unique. A UMI can include one or more specific polynucleotides sequences, one or more random nucleic acid and/or amino acid sequences, and/or one or more synthetic nucleic acid and/or amino acid sequences.

In some embodiments, the UMI is a nucleic acid sequence that does not substantially hybridize to analyte nucleic acid molecules in a biological sample. In some embodiments, the UMI has less than 90% sequence identity (e.g., less than 80%, 70%, 60%, 50%, or less than 40% sequence identity) to the nucleic acid sequences across a substantial part (e.g., 80% or more) of the nucleic acid molecules in the biological sample.

The UMI can include from about 6 to about 20 or more nucleotides within the sequence of capture probes, e.g., barcoded oligonucleotides in an array generated using a method disclosed herein. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 15, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter. These nucleotides can be contiguous, i.e., in a single stretch of adjacent nucleotides, or they can be separated into two or more separate subsequences that are separated by 1 or more nucleotides. Separated UMI subsequences can be from about 4 to about 16 nucleotides in length. In some embodiments, the UMI subsequence can be about 4, 5, 6, 7, 8, 9, 10, 11, 12, 13, 14, 15, 16 nucleotides or longer. In some embodiments, the UMI subsequence can be at least about 4, 5, 6, 7, 8, 9, 10, 11, 12, 13, 14, 15, 16 nucleotides or longer. In some embodiments, the UMI subsequence can be at most about 4, 5, 6, 7, 8, 9, 10, 11, 12, 13, 14, 15, 16 nucleotides or shorter.

In some embodiments, a UMI is attached to other parts of the oligonucleotide in a reversible or irreversible manner. In some embodiments, a UMI is added to, for example, a fragment of a DNA or RNA sample before sequencing of the analyte. In some embodiments, a UMI allows for identification and/or quantification of individual sequencing-reads. In some embodiments, a UMI is used as a fluorescent barcode for which fluorescently labeled oligonucleotide probes hybridize to the UMI.

In some embodiments, a method provided herein further comprises a step of providing the substrate. A wide variety of different substrates can be used for the foregoing purposes. In general, a substrate can be any suitable support material. The substrate may comprise materials of one or more of the IUPAC Groups 4, 6, 11, 12, 13, 14, and 15 elements, plastic material, silicon dioxide, glass, fused silica, mica, ceramic, or metals deposited on the aforementioned substrates. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, quartz, metals, inorganic glasses, optical fiber bundles, and polymers, such as polystyrene, cyclic olefin copolymers (COCs), cyclic olefin polymers (COPs), polypropylene, polyethylene and polycarbonate. In some embodiments, the substrate is a glass substrate.

A substrate can be of any desired shape. For example, a substrate can be typically a thin (e.g., sub-centimeter), flat shape (e.g., square, rectangle or a circle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip, wafer, die, or a slide such as a microscope slide).

In some embodiments, the surface of the substrate is coated. In some embodiments, the surface of the substrate is coated with a photoresist.

In some aspects, disclosed herein is a method for generating a molecular array, comprising irradiating a substrate through a first photomask comprising an opening corresponding to a region of a plurality of regions on the substrate, wherein a first oligonucleotide of at least four nucleotides in length is attached to oligonucleotide molecules in the region to generate extended oligonucleotide molecules. Multiple cycles of the irradiation and oligonucleotide attachment can be performed, one cycle for each of the plurality of regions, by translating the first photomask across the substrate until all regions have received the first oligonucleotide. In some embodiments, the method can further comprise irradiating the substrate through a second photomask comprising multiple openings corresponding to a set of sub-regions each of which is in one of the regions, wherein a second oligonucleotide of at least four nucleotides in length is attached to the extended oligonucleotide molecules in the set of sub-regions to generate further extended oligonucleotide molecules. Multiple cycles of the irradiation and oligonucleotide attachment can be performed, one cycle for each set of sub-regions, by translating the second photomask across the substrate until all sub-regions of all regions have received the second oligonucleotide, thereby providing on the substrate an array comprising oligonucleotide molecules. In some embodiments, the method can further comprise irradiating the substrate through a third photomask comprising multiple openings corresponding to a set of sub-sub-regions each of which is in one of the sub-regions, wherein a third oligonucleotide of at least four nucleotides in length is attached to the further extended oligonucleotide molecules in the set of sub-sub-regions to generate even further extended oligonucleotide molecules. Again, multiple cycles of the irradiation and oligonucleotide attachment are performed, one cycle for each set of sub-sub-regions, by translating the third photomask across the substrate until all sub-sub-regions of all sub-regions of all regions have received the third oligonucleotide. The process can be repeated to generate finer and finer features on the substrate.

### B. Methods of Removing Polynucleotides

In some embodiments, oligonucleotides that are uncaged and do not receive a ligated oligonucleotide could receive the incorrect barcode during the next cycle or round. In order to prevent generating the wrong barcode at the wrong location on an array (e.g., feature or spot), unligated oligonucleotides may be rendered unavailable for hybridization and/or ligation, e.g., the unligated oligonucleotides can be capped and/or removed. In some embodiments, the oligonucleotides are modified at the 3′. Non-limiting examples of 3′ modifications include dideoxy C-3′ (3′-ddC), 3′ inverted dT, 3′ C3 spacer, 3′Amino, and 3′ phosphorylation.

In one aspect, disclosed herein is a method for providing differentially barcoded polynucleotides, comprising: (i) rendering a polynucleotide immobilized on a substrate unavailable for ligation, wherein the substrate has immobilized thereon: a first polynucleotide comprising a first barcode comprising a first photo-cleavable moiety, a second polynucleotide comprising a second photo-cleavable moiety, and a third polynucleotide available for ligation, wherein said first photo-cleavable moiety and said second photo-cleavable moiety block ligation to said first polynucleotide and said second polynucleotide, respectively, and wherein the third polynucleotide is rendered unavailable for ligation; (ii) irradiating the second polynucleotide with light while the first polynucleotide is masked, thereby rendering said second polynucleotide available for ligation whereas said first polynucleotide remains unavailable for ligation; (iii) ligating a second barcode to said second polynucleotide, wherein said second barcode is not ligated to said first polynucleotide, thereby providing differentially barcoded polynucleotides on the substrate.

In one aspect, disclosed herein is a method for providing differentially barcoded polynucleotides, comprising: (i) irradiating a subset of a plurality of polynucleotides immobilized on a substrate, wherein each of the polynucleotides comprises a first photo-cleavable moiety that blocks ligation to the polynucleotide, wherein after said irradiation the substrate has at least: a first polynucleotide which is rendered available for ligation due to photo-cleavage of the first photo-cleavable moiety, a second polynucleotide which retains said first photo-cleavable moiety and is not available for ligation, and a third polynucleotide which is rendered available for ligation due to photo-cleavage of the first photo-cleavable moiety; (ii) ligating a first barcode comprising a second photo-cleavable moiety to said first polynucleotide, wherein said first barcode is not ligated to said second or third polynucleotide, wherein after said ligation: said first polynucleotide comprises said first barcode comprising said second photo-cleavable moiety and is rendered unavailable for ligation, said second polynucleotide retains said first photo-cleavable moiety and is not available for ligation, and said third polynucleotide remains available for ligation; (iii) rendering said third polynucleotide unavailable for ligation, whereby irradiation of said second polynucleotide allows ligation of a second barcode thereto, whereas said second barcode is not ligated to said third polynucleotide, thereby providing differentially barcoded polynucleotides on the substrate.

In one other aspect, disclosed herein is a method for providing a barcoded polynucleotide, comprising: (i) irradiating a subset of a plurality of polynucleotides immobilized on a substrate, wherein each of the polynucleotides comprises a first photo-cleavable moiety that blocks ligation to the polynucleotide, wherein after said irradiation the substrate has at least: a first polynucleotide which is rendered available for ligation due to photo-cleavage of the first photo-cleavable moiety, a second polynucleotide which retains said first photo-cleavable moiety and is not available for ligation, and a third polynucleotide which is rendered available for ligation due to photo-cleavage of the first photo-cleavable moiety; (ii) ligating a first barcode which is nuclease resistant to said first polynucleotide, wherein said first barcode is not ligated to said second or third polynucleotide, wherein after said ligation: said first polynucleotide comprises said first barcode which is nuclease resistant, said second polynucleotide retains said first photo-cleavable moiety and is not available for ligation, and said third polynucleotide remains available for ligation; (iii) contacting said polynucleotides with a nuclease, whereby said second and third polynucleotides are cleaved from the substrate and said first polynucleotide is not cleaved; and (iv) attaching a universal adaptor to said first barcode, wherein said universal adaptor comprises a second photo-cleavable moiety that blocks ligation to the polynucleotide, thereby providing said first polynucleotide which is barcoded.

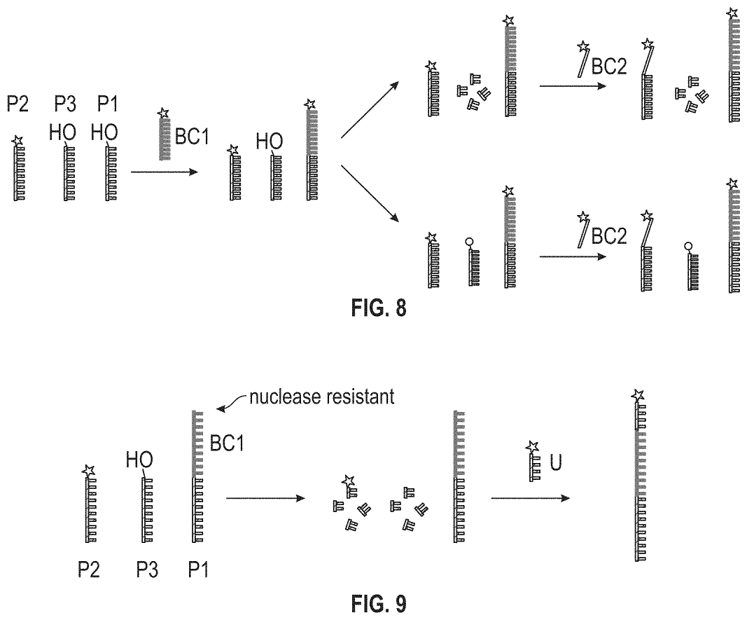

In one aspect, provided herein is a method for providing an array of polynucleotides, comprising attaching a first barcode to a first polynucleotide immobilized on a substrate. For example, as shown in FIG. 6, a substrate (not shown) has immobilized thereon (i) a first polynucleotide P1, (ii) a second polynucleotide P2 comprising a photo-cleavable moiety that inhibits or blocks hybridization and/or ligation to the second polynucleotide, and (iii) a third polynucleotide P3, where both P1 and P3 are available for hybridization and/or ligation. A first barcode BC1 is then attached to P1, wherein BC1 comprises a photo-cleavable moiety that inhibits or blocks hybridization and/or ligation. After the attaching step, the substrate has immobilized thereon (i) P1 barcoded with BC1 comprising the photo-cleavable moiety, (ii) P2, which retains the photo-cleavable moiety, and (iii) P3, which is available for hybridization and/or ligation, but has not received a barcode in the attaching step. If P3 is not removed, it may receive a barcode in the next attaching step. In other words, instead of correctly receiving barcode BC1, P3 if not removed would receive a barcode BC2 which is not correct. Thus, prior to the attaching the next barcode, P3 should be rendered unavailable for hybridization and/or ligation, for example, through exonuclease digestion of P3, while P1 and P2 are protected by the photo-cleavable moiety and are not digested by the exonuclease. Alternatively, as shown in FIG. 8, the 3′ of the unprotected polynucleotide P3 may be capped (see, filled circle), e.g., to prevent future ligation to P3. In some embodiments, the capping comprises adding a 3′ dideoxy, a non-ligating 3′ phosphoramidate, or a triphenylmethyl (trityl) group to the 3′ of unprotected polynucleotide molecules. In some embodiments, the addition is catalyzed by an enzyme. In some embodiments, the enzyme is a template-independent polymerase, such as a terminal deoxynucleotidyl transferase (TdT) or a poly(A) polymerase. In some embodiments, capping by the trityl group is removed with a mild acid.

In another aspect, the method for providing an array of polynucleotides comprises irradiating a substrate with light. For example, as shown in FIG. 7, a substrate (not shown) has immobilized thereon (i) a first polynucleotide P1 comprising a first barcode BC1 comprising a photo-cleavable moiety, (ii) a second polynucleotide P2 comprising a photo-cleavable moiety, and (iii) a third polynucleotide P3 available for hybridization and/or ligation. The photo-cleavable moiety inhibits or blocks hybridization and/or ligation to P1 and P2. As in FIG. 6, if P3 is not removed, it may receive a barcode in the next attaching step. P3 may be rendered unavailable for hybridization and/or ligation, for example, through exonuclease digestion of P3 (while P1 and P2 are protected by the photo-cleavable moiety) and/or 3′ capping to prevent future ligation to P3. As such, when the next barcode BC2 is attached (while P1-BC1 is photomasked) in FIG. 7, BC2 is correctly attached to P2 but not P3.

In one aspect, provided herein is a method for providing an array of polynucleotides, comprising attaching a first barcode to a first polynucleotide immobilized on a substrate, and irradiating the substrate with light. For example, as shown in FIG. 8, a substrate (not shown) has immobilized thereon (i) a first polynucleotide P1, (ii) a second polynucleotide P2 comprising a photo-cleavable moiety that inhibits or blocks hybridization and/or ligation to the second polynucleotide, and (iii) a third polynucleotide P3, where both P1 and P3 are available for hybridization and/or ligation. A first barcode BC1 is then attached to P1, wherein BC1 comprises a photo-cleavable moiety that inhibits or blocks hybridization and/or ligation. Prior to the attaching the next barcode, P3 is rendered unavailable for hybridization and/or ligation, for example, through exonuclease digestion of P3 (while P1 and P2 are protected by the photo-cleavable moiety) and/or 3′ capping to prevent future ligation to P3. Then, the substrate is exposed to light, where P2 is deprotected while P1 barcoded with BC1 remains protected (e.g., P1 barcoded with BC1 is photomasked while P2 is not). BC2 is correctly ligated to P2, and not to P3 or the BC1-barcoded P1.

In another aspect, provided herein is a method for providing an array of polynucleotides, comprising attaching a barcode which is nuclease resistant to a polynucleotide immobilized on a substrate. For example, as shown in FIG. 9, a nuclease resistant barcode BC1 is attached to a first polynucleotide P1 immobilized on a substrate (not shown). The substrate has immobilized thereon (i) P1 with barcode BC1, (ii) a second polynucleotide P2 comprising a first photo-cleavable moiety that inhibits or blocks hybridization and/or ligation, and (iii) a third polynucleotide P3 available for hybridization and/or ligation. In some embodiments, the method further comprises rendering the third polynucleotide (P3) unavailable for hybridization and/or ligation. For example, P3 is rendered unavailable for hybridization and/or ligation by nuclease digestion, whereas P1 is rendered nuclease resistant due to its attachment to BC1. In some embodiments, the second polynucleotide is also digested by the nuclease. An adaptor (U) may be attached to BC1 which is in turn attached to P1, and U may comprise a photo-cleavable moiety that inhibits or blocks hybridization and/or ligation. In some embodiments, the adaptor is a universal adaptor, for example, for hybridization and/or ligation of a round 2 barcode or an oligonucleotide cassette comprising the round 2 barcode.

C. Compositions and Methods of Use

Also provided are compositions produced according to the methods described herein. These compositions include nucleic acid molecules and complexes, such as hybridization complexes, and kits and articles of manufacture (such as arrays) comprising such molecules and complexes.

In one aspect, provided herein is a hybridization complex, comprising: a first polynucleotide immobilized on a substrate; a first splint hybridized to one end of the first polynucleotide and one end of a first barcode; and the first barcode which comprise a first photo-cleavable moiety that inhibits or blocks hybridization and/or ligation to the other end of the first barcode.

In another aspect, provided herein is a composition, comprising the hybridization complex disclosed herein and a substrate. In some embodiments, the composition further comprises a second polynucleotide immobilized on the substrate, wherein the second polynucleotide comprises a second photo-cleavable moiety that inhibits or blocks hybridization and/or ligation to second polynucleotide.

In some embodiments, the composition further comprises a second hybridization complex comprising: a second polynucleotide immobilized on the substrate; a second splint hybridized to one end of the second polynucleotide and one end of a second barcode; and the second barcode which comprise a second photo-cleavable moiety that inhibits or blocks hybridization and/or ligation to the other end of the second barcode.

In any of the embodiments herein, the first polynucleotide and the second polynucleotide can be of the same nucleic acid sequence or different nucleic acid sequences.

In any of the embodiments herein, the first barcode and the second barcode can be of the same nucleic acid sequence or different nucleic acid sequences.

In any of the embodiments herein, the first splint and the second splint can be of the same nucleic acid sequence or different nucleic acid sequences.

In any of the embodiments herein, the first polynucleotide and/or the second polynucleotide can be single stranded.

In any of the embodiments herein, the first polynucleotide and/or the second polynucleotide can be DNA oligonucleotides.

In any of the embodiments herein, the first photo-cleavable moiety and/or the second photo-cleavable moiety can comprise a photo-caged nucleobase, such as a photo-caged deoxythymidine (dT). In some embodiments, the photo-cleavable moiety comprises the following structure:

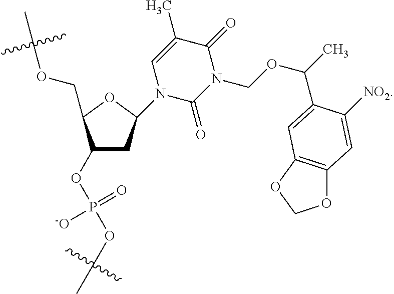

In any of the embodiments herein, the first photo-cleavable moiety and/or the second photo-cleavable moiety can comprise a photo-cleavable linker, for example, one that forms a hairpin. In some embodiments, the photo-cleavable moiety comprises the following structure:

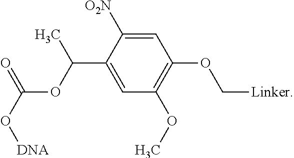

In any of the embodiments herein, the first photo-cleavable moiety and/or the second photo-cleavable moiety can comprise a photo-caged 3-hydroxyl group. In some embodiments, the photo-cleavable moiety comprises the following structure:

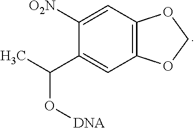

In some embodiments, the first photo-cleavable moiety and/or the second photo-cleavable moiety can comprise a photo-cleavable spacer, for example, a spacer that lacks a 5′ phosphate for ligation. In some embodiments, the photo-cleavable moiety comprises the following structure:

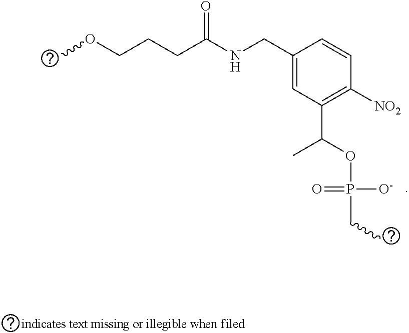

In any of the embodiments herein, the first photo-cleavable moiety and/or the second photo-cleavable moiety can comprise a photo-cleavable polymer.

Also provided herein are arrays comprising any one or more of the molecules, complexes, and/or compositions disclosed herein. Typically, an array includes at least two distinct nucleic acids that differ by monomeric sequence immobilized thereon, e.g., covalently to, different and known locations on the substrate surface. In certain embodiments, each distinct nucleic acid sequence of the array is typically present as a composition of multiple copies of the polymer on the substrate surface, e.g. as a spot on the surface of the substrate. The number of distinct nucleic acid sequences, and hence spots or similar structures, present on the array may vary, but is generally at least, usually at least 5 and more usually at least 10, where the number of different spots on the array may be as a high as 50, 100, 500, 1000, 10,000, 1,000,000, 10,000,000 or higher, depending on the intended use of the array. The spots of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like. The density of spots present on the array surface may vary, but is generally at least about 10 and usually at least about 100 spots/cm², where the density may be as high as 10⁶ or higher, or about 10⁵ spots/cm². In other embodiments, the polymeric sequences are not arranged in the form of distinct spots, but may be positioned on the surface such that there is substantially no space separating one polymer sequence/feature from another. The density of nucleic acids within an individual feature on the array may be as high as 1,000, 10,000, 25,000, 50,000, 100,000, 500,000, 1,000,000, or higher per square micron depending on the intended use of the array.

In some embodiments, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini, e.g. the 3′ or 5′ terminus.

Arrays can be used to measure large numbers of analytes simultaneously. In some embodiments, oligonucleotides are used, at least in part, to create an array. For example, one or more copies of a single species of oligonucleotide (e.g., capture probe) can correspond to or be directly or indirectly attached to a given feature in the array. In some embodiments, a given feature in the array includes two or more species of oligonucleotides (e.g., capture probes). In some embodiments, the two or more species of oligonucleotides (e.g., capture probes) attached directly or indirectly to a given feature on the array include a common (e.g., identical) spatial barcode.

In some embodiments, an array can include a capture probe attached directly or indirectly to the substrate. The capture probe can include a capture domain (e.g., a nucleotide sequence) that can specifically bind (e.g., hybridize) to a target analyte (e.g., mRNA, DNA, or protein) within a sample. In some embodiments, the binding of the capture probe to the target (e.g., hybridization) can be detected and quantified by detection of a visual signal, e.g., a fluorophore, a heavy metal (e.g., silver ion), or chemiluminescent label, which has been incorporated into the target. In some embodiments, the intensity of the visual signal correlates with the relative abundance of each analyte in the biological sample. Since an array can contain thousands or millions of capture probes (or more), an array can interrogate many analytes in parallel. In some embodiments, the binding (e.g., hybridization) of the capture probe to the target can be detected and quantified by creation of a molecule (e.g., cDNA from captured mRNA generated using reverse transcription) that is removed from the array, and processed downstream (e.g., sequenced).

Kits for use in analyte detection assays are provided. In some embodiments, the kit at least includes an array disclosed herein. The kits may further include one or more additional components necessary for carrying out an analyte detection assay, such as sample preparation reagents, buffers, labels, and the like. As such, the kits may include one or more containers such as tubes, vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the subject array assay devices for carrying out an array based assay. The instructions may be printed on a substrate, such as paper or plastic, etc. As such, the instructions may be present in the kits as a package insert, in the labeling of the container of the kit or components thereof (i.e., associated with the packaging or sub-packaging) etc.

The subject arrays find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label present on the analyte, etc., and/or through sequencing of one or more components of the binding complex or a product thereof. The presence of the analyte in the sample is then deduced from the detection of binding complexes on the substrate surface, or sequence detection and/or analysis (e.g., by sequencing) on molecules indicative of the formation of the binding complex. In some embodiments, RNA molecules (e.g., mRNA) from a sample are captured by oligonucleotides (e.g., probes comprising a barcode and a poly(dT) sequence) on an array prepared by a method disclosed herein, cDNA molecules are generated via reverse transcription of the captured RNA molecules, and the cDNA molecules (e.g., a first strand cDNA) or portions or products (e.g., a second strand cDNA synthesized using a template switching oligonucleotide) thereof can be separated from the array and sequenced. Sequencing data obtained from molecules prepared on the array can be used to deduce the presence/absence or an amount of the RNA molecules in the sample.

Specific analyte detection applications of interest include hybridization assays in which the nucleic acid arrays of the present disclosure are employed. In these assays, a sample of target nucleic acids or a tissue section is first prepared, where preparation may include labeling of the target nucleic acids with a label, e.g., a member of signal producing system. Following sample preparation, the sample is contacted with the array under hybridization conditions, whereby complexes are formed between target nucleic acids that are complementary to probe sequences attached to the array surface. The formation and/or presence of hybridized complexes is then detected, e.g., by analyzing molecules that are generated following the formation of the hybridized complexes, such as cDNA or a second strand generated from an RNA captured on the array. Specific hybridization assays of interest which may be practiced using the subject arrays include: gene discovery assays, differential gene expression analysis assays; nucleic acid sequencing assays, single nucleotide polymorphism (SNP) assays, copy number variation (CNV) assays, and the like.

### Spatial Analysis

In some aspects, provided herein is a method for construction of a hybridization complex or an array comprising nucleic acid molecules and complexes. Oligonucleotide probe for capturing analytes which may be generated using a method disclosed herein, for example, using two, three, four, or more rounds of hybridization and ligation shown in FIG. 4.

In some embodiments, the oligonucleotide probe for capturing analytes may be generated from an existing array with a ligation strategy. In some embodiments, an array containing a plurality of oligonucleotides (e.g., in situ synthesized oligonucleotides) can be modified to generate a variety of oligonucleotide probes. The oligonucleotides can include various domains such as, spatial barcodes, UMIs, functional domains (e.g., sequencing handle), cleavage domains, and/or ligation handles.

A “spatial barcode” may comprise a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate. A spatial barcode can be part of a capture probe on an array generated herein. A spatial barcode can also be a tag attached to an analyte (e.g., a nucleic acid molecule) or a combination of a tag in addition to an endogenous characteristic of the analyte (e.g., size of the analyte or end sequence(s)). A spatial barcode can be unique. In some embodiments where the spatial barcode is unique, the spatial barcode functions both as a spatial barcode and as a unique molecular identifier (UMI), associated with one particular capture probe. Spatial barcodes can have a variety of different formats. For example, spatial barcodes can include polynucleotide spatial barcodes; random nucleic acid and/or amino acid sequences; and synthetic nucleic acid and/or amino acid sequences. In some embodiments, a spatial barcode is attached to an analyte in a reversible or irreversible manner. In some embodiments, a spatial barcode is added to, for example, a fragment of a DNA or RNA sample before sequencing of the sample. In some embodiments, a spatial barcode allows for identification and/or quantification of individual sequencing-reads. In some embodiments, a spatial barcode is a used as a fluorescent barcode for which fluorescently labeled oligonucleotide probes hybridize to the spatial barcode.

In some embodiments, a spatial array is generated after ligating capture domains (e.g., poly(T) or gene specific capture domains) to the oligonucleotide molecule (e.g., generating capture oligonucleotides). The spatial array can be used with any of the spatial analysis methods described herein. For example, a biological sample (e.g., a tissue section) can be provided to the generated spatial array. In some embodiments, the biological sample is permeabilized. In some embodiments, the biological sample is permeabilized under conditions sufficient to allow one or more analytes present in the biological sample to interact with the capture probes of the spatial array. After capture of analytes from the biological sample, the analytes can be analyzed (e.g., reverse transcribed, amplified, and/or sequenced) by any of the variety of methods described herein.

Sequential hybridization/ligation of various domains can be used to generate an oligonucleotide probe for capturing analytes, by a photo-hybridization/ligation method described herein. For example, an oligonucleotide can be immobilized on a substrate (e.g., an array) and may comprise a functional sequence such as a primer sequence. In some embodiments, the primer sequence is a sequencing handle that comprises a primer binding site for subsequent processing. The primer sequence can generally be selected for compatibility with any of a variety of different sequencing systems, e.g., 454 Sequencing, Ion Torrent Proton or PGM, Illumina X10, PacBio, Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Roche 454 sequencing, Ion Torrent Proton or PGM sequencing, Illumina X10 sequencing, PacBio SMRT sequencing, and Oxford Nanopore sequencing. Further, in some embodiments, functional sequences can be selected for compatibility with other sequencing systems, including non-commercialized sequencing systems.

In some embodiments, in a first cycle of photo-hybridization/ligation, an oligonucleotide comprising a part of a barcode (e.g., part A of the barcode) is attached to the oligonucleotide molecule comprising the primer (e.g., R1 primer). In some embodiments, the barcode part can be common to all of the oligonucleotide molecules in a given feature. In some embodiments, the barcode part can be common to all of the oligonucleotide molecules in multiple substrate regions (e.g., features) in the same cycle. In some embodiments, the barcode part can be different for oligonucleotide molecules in different substrate regions (e.g., features) in different cycle. In some embodiments, a splint with a sequence complementary to a portion of the primer of the immobilized oligonucleotide and an additional sequence complementary to a portion of the oligonucleotide comprising the part of the barcode (e.g., part A of the barcode) facilitates the ligation of the immobilized oligonucleotide and the oligonucleotide comprising the barcode part. In some embodiments, the splint for attaching the part of the barcode of various sequences to different substrate regions (e.g., features) is common among the cycles of the same round. In some embodiments, the splint for attaching the part of the barcode of various sequences to different substrate regions (e.g., features) can be different among the cycles of the same round. In some embodiments, the splint for attaching the part of the barcode may comprise a sequence complementary to the part or a portion thereof.

A second cycle of photo-hybridization/ligation can involve the addition of another oligonucleotide comprising another part of a barcode (e.g., part B of the barcode) to the immobilized oligonucleotide molecule comprising the primer and part A of the barcode. As shown in the figure, in some embodiments, a splint with a sequence complementary to a portion of the immobilized oligonucleotide comprising part A of the barcode and an additional sequence complementary to a portion of the oligonucleotide comprising part B of the barcode facilitates the ligation of the oligonucleotide comprising part B and the immobilized oligonucleotide comprising part A. In some embodiments, the splint for attaching part B of various sequences to different substrate regions (e.g., features) is common among the cycles of the same round. In some embodiments, the splint for attaching part B to different substrate regions (e.g., features) can be different among the cycles of the same round. In some embodiments, the splint for attaching part B may comprise a sequence complementary to part B or a portion thereof and/or a sequence complementary to part A or a portion thereof.

A third cycle of photo-hybridization/ligation can involve the addition of another oligonucleotide comprising another part of a barcode (e.g., part C of the barcode), added to the immobilized oligonucleotide molecule comprising the primer, part A, and part B. In some embodiments, a splint with a sequence complementary to a portion of the immobilized oligonucleotide molecule comprising part B and an additional sequence complementary to a portion of the oligonucleotide comprising part C facilitates the ligation of the immobilized oligonucleotide molecule comprising part B and the oligonucleotide comprising part C. In some embodiments, the splint for attaching part part C of various sequences to different substrate regions (e.g., features) is common among the cycles of the same round. In some embodiments, the splint for attaching part C to different substrate regions (e.g., features) can be different among the cycles of the same round. In some embodiments, the splint for attaching part C may comprise a sequence complementary to part C or a portion thereof and/or a sequence complementary to part B or a portion thereof.

A fourth cycle of photo-hybridization/ligation may be performed, which involves the addition of another oligonucleotide comprising another part of a barcode (e.g., part D of the barcode), added to the immobilized oligonucleotide molecule comprising the primer, part A, part B, and part C. In some embodiments, a splint with a sequence complementary to a portion of the immobilized oligonucleotide molecule comprising part C and an additional sequence complementary to a portion of the oligonucleotide comprising part D facilitates the ligation. In some embodiments, the splint for attaching part part D of various sequences to different substrate regions (e.g., features) is common among the cycles of the same round. In some embodiments, the splint for attaching part D to different substrate regions (e.g., features) can be different among the cycles of the same round. In some embodiments, the splint for attaching part D may comprise a sequence complementary to part D or a portion thereof and/or a sequence complementary to part C or a portion thereof. In some embodiments, an oligonucleotide comprising part D further comprises a UMI and/or a capture domain.

In particular embodiments, provided herein are kits and compositions for spatial array-based analysis of biological samples. Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of each analyte within the biological sample. The spatial location of each analyte within the biological sample is determined based on the feature to which each analyte is bound on the array, and the feature's relative spatial location within the array. In some embodiments, the array of features on a substrate comprises a spatial barcode that corresponds to the feature's relative spatial location within the array. Each spatial barcode of a feature may further comprise a fluorophore, to create a fluorescent hybridization array. A feature may comprise UMIs that are generally unique per nucleic acid molecule in the feature—so the number of unique molecules can be estimated, as opposed to an artifact in experiments or PCR amplification bias that drives amplification of smaller, specific nucleic acid sequences.

In particular embodiments, the kits and compositions for spatial array-based analysis provide for the detection of differences in an analyte level (e.g., gene and/or protein expression) within different cells in a tissue of a mammal or within a single cell from a mammal. For example, the kits and compositions can be used to detect the differences in analyte levels (e.g., gene and/or protein expression) within different cells in histological slide samples (e.g., tissue section), the data from which can be reassembled to generate a three-dimensional map of analyte levels (e.g., gene and/or protein expression) of a tissue sample obtained from a mammal, e.g., with a degree of spatial resolution (e.g., single-cell scale resolution).

In some embodiments, an array generated using a method disclosed herein can be used in array-based spatial analysis methods which involve the transfer of one or more analytes from a biological sample to an array of features on a substrate, each of which is associated with a unique spatial location on the array. Subsequent analysis of the transferred analytes includes determining the identity of the analytes and the spatial location of each analyte within the sample. The spatial location of each analyte within the sample is determined based on the feature to which each analyte is bound in the array, and the feature's relative spatial location within the array.

There are at least two general methods to associate a spatial barcode with one or more neighboring cells, such that the spatial barcode identifies the one or more cells, and/or contents of the one or more cells, as associated with a particular spatial location. One general method is to drive target analytes out of a cell and towards the spatially-barcoded array. In some embodiments, the spatially-barcoded array populated with capture probes is contacted with a sample (e.g., a tissue section or a population of single cells), and the sample is permeabilized, allowing the target analyte to migrate away from the sample and toward the array. The target analyte interacts with a capture probe on the spatially-barcoded array. Once the target analyte hybridizes/is bound to the capture probe, the sample is optionally removed from the array and the capture probes are analyzed in order to obtain spatially-resolved analyte information.

Another general method is to cleave the spatially-barcoded capture probes from an array, and drive the spatially-barcoded capture probes towards and/or into or onto the sample. In some embodiments, the spatially-barcoded array populated with capture probes is contacted with a sample. The spatially-barcoded capture probes are cleaved and then interact with cells within the provided sample. The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed (e.g., by sequencing) to obtain spatially-resolved information about the tagged cell.

Sample preparation may include placing the sample on a slide, fixing the sample, and/or staining the sample for imaging. The stained sample may be imaged on the array using both brightfield (to image the sample hematoxylin and eosin stain) and/or fluorescence (to image features) modalities. In some embodiments, target analytes are then released from the sample and capture probes forming the spatially-barcoded array hybridize or bind the released target analytes. The sample is then removed from the array and the capture probes cleaved from the array. The sample and array are then optionally imaged a second time in one or both modalities (brightfield and fluorescence) while the analytes are reverse transcribed into cDNA, and an amplicon library is prepared and sequenced. In some embodiments, the two sets of images can then be spatially-overlaid in order to correlate spatially-identified sample information. When the sample and array are not imaged a second time, a spot coordinate file may be supplied. The spot coordinate file can replace the second imaging step. Further, amplicon library preparation can be performed with a unique PCR adapter and sequenced.

In some embodiments, a spatially-labelled array on a substrate is used, where capture probes labelled with spatial barcodes are clustered at areas called features. The spatially-labelled capture probes can include a cleavage domain, one or more functional sequences, a spatial barcode, a unique molecular identifier, and a capture domain. The spatially-labelled capture probes can also include a 5′ end modification for reversible attachment to the substrate. The spatially-barcoded array is contacted with a sample, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, wherein the sample is sandwiched between the diffusion-resistant medium and/or barrier and the array-containing substrate. The analytes are migrated toward the spatially-barcoded capture array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatially-barcoded capture probes, the capture probes can hybridize or otherwise bind a target analyte. The sample can be optionally removed from the array.

Adapters and assay primers can be used to allow the capture probe or the analyte capture agent to be attached to any suitable assay primers and used in any suitable assays. A capture probe that includes a spatial barcode can be attached to a bead that includes a poly(dT) sequence. A capture probe including a spatial barcode and a poly(T) sequence can be used to assay multiple biological analytes as generally described herein (e.g., the biological analyte includes a poly(A) sequence or is coupled to or otherwise is associated with an analyte capture agent comprising a poly(A) sequence as the analyte capture sequence).

The capture probes can be optionally cleaved from the array, and the captured analytes can be spatially-tagged by performing a reverse transcriptase first strand cDNA reaction. A first strand cDNA reaction can be optionally performed using template switching oligonucleotides. For example, a template switching oligonucleotide can hybridize to a poly(C) tail added to a 3′end of the cDNA by a reverse transcriptase enzyme. The original mRNA template and template switching oligonucleotide can then be denatured from the cDNA and the barcoded capture probe can then hybridize with the cDNA and a complement of the cDNA can be generated. The first strand cDNA can then be purified and collected for downstream amplification steps. The first strand cDNA can be amplified using PCR, wherein forward and reverse primers flank the spatial barcode and target analyte regions of interest, generating a library associated with a particular spatial barcode. In some embodiments, the cDNA comprises a sequencing by synthesis (SBS) primer sequence. The library amplicons are sequenced and analyzed to decode spatial information.

In some embodiments, the sample is removed from the spatially-barcoded array and the spatially-barcoded capture probes are removed from the array for barcoded analyte amplification and library preparation. In some embodiments, the sample is removed from the spatially-barcoded array prior to removal of the spatially-barcoded capture probes from the array. Another embodiment includes performing first strand synthesis using template switching oligonucleotides on the spatially-barcoded array without cleaving the capture probes. Once the capture probes capture the target analyte(s), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, A-tailing, and indexing PCR steps, and then sequenced.

### Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used in this specification and the appended claims, the singular forms “a,” “an” and “the” include plural referents unless the context clearly dictates otherwise. Thus, for example, reference to “a molecule” includes a plurality of such molecules, and the like.

A sample such as a biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

The term “barcode” comprises a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell scale resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (i.e., can be oligoribonucleotides) and/or deoxyribonucleotide monomers (i.e., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

An “adaptor,” an “adapter,” and a “tag” are terms that are used interchangeably in this disclosure, and refer to species that can be coupled to a polynucleotide sequence (in a process referred to as “tagging”) using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences.

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

A “proximity ligation” is a method of ligating two (or more) nucleic acid sequences that are in proximity with each other through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference).

A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

A “splint” is an oligonucleotide that, when hybridized to other polynucleotides, acts as a “splint” to position the polynucleotides next to one another so that they can be ligated together. In some embodiments, the splint is DNA or RNA. The splint can include a nucleotide sequence that is partially complimentary to nucleotide sequences from two or more different oligonucleotides. In some embodiments, the splint assists in ligating a “donor” oligonucleotide and an “acceptor” oligonucleotide. In general, an RNA ligase, a DNA ligase, or another other variety of ligase is used to ligate two nucleotide sequences together

In some embodiments, the splint is between 6 and 50 nucleotides in length, e.g., between 6 and 45, 6 and 40, 6 and 35, 6 and 30, 6 and 25, or 6 and 20 nucleotides in length. In some embodiments, the splint is between 10 and 50 nucleotides in length, e.g., between 10 and 45, 10 and 40, 10 and 35, 10 and 30, 10 and 25, or 10 and 20 nucleotides in length. In some embodiments, the splint is between 15 and 50, 15 and 45, 15 and 40, 15 and 35, 15 and 30, 15 and 30, or 15 and 25 nucleotides in length.

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis. Similarly, a 3′ polyadenylated tail of an mRNA transcript that hybridizes to a poly (dT) sequence (e.g., capture domain) can be used as a template for single-strand synthesis of a corresponding cDNA molecule.

As used herein, the term “substrate” generally refers to a substance, structure, surface, material, means, or composition, which comprises a nonbiological, synthetic, nonliving, planar, spherical or flat surface. The substrate may include, for example and without limitation, semiconductors, synthetic metals, synthetic semiconductors, insulators and dopants; metals, alloys, elements, compounds and minerals; synthetic, cleaved, etched, lithographed, printed, machined and microfabricated slides, wafers, devices, structures and surfaces; industrial polymers, plastics, membranes; silicon, silicates, glass, metals and ceramics; wood, paper, cardboard, cotton, wool, cloth, woven and nonwoven fibers, materials and fabrics; nanostructures and microstructures. The substrate may comprise an immobilization matrix such as but not limited to, insolubilized substance, solid phase, surface, layer, coating, woven or nonwoven fiber, matrix, crystal, membrane, insoluble polymer, plastic, glass, biological or biocompatible or bioerodible or biodegradable polymer or matrix, microparticle or nanoparticle. Other examples may include, for example and without limitation, monolayers, bilayers, commercial membranes, resins, matrices, fibers, separation media, chromatography supports, polymers, plastics, glass, mica, gold, beads, microspheres, nanospheres, silicon, gallium arsenide, organic and inorganic metals, semiconductors, insulators, microstructures and nanostructures. Microstructures and nanostructures may include, without limitation, microminiaturized, nanometer-scale and supramolecular probes, tips, bars, pegs, plugs, rods, sleeves, wires, filaments, and tubes.

A “feature” is an entity that acts as a support or repository for various molecular entities used in sample analysis. In some embodiments, some or all of the features in an array are functionalized for analyte capture. In some embodiments, functionalized features include one or more capture probe(s). Examples of features include, but are not limited to, a bead, a spot of any two- or three-dimensional geometry (e.g., an ink jet spot, a masked spot, a square on a grid), a well, and a hydrogel pad. In some embodiments, features are directly or indirectly attached or fixed to a substrate. In some embodiments, the features are not directly or indirectly attached or fixed to a substrate, but instead, for example, are disposed within an enclosed or partially enclosed three dimensional space (e.g., wells or divots).

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “template” as used herein generally refers to individual polynucleotide molecules from which another nucleic acid, including a complementary nucleic acid strand, can be synthesized by a nucleic acid polymerase. In addition, the template can be one or both strands of the polynucleotides that are capable of acting as templates for template-dependent nucleic acid polymerization catalyzed by the nucleic acid polymerase. Use of this term should not be taken as limiting the scope of the present disclosure to polynucleotides which are actually used as templates in a subsequent enzyme-catalyzed polymerization reaction. The template can be an RNA or DNA. The template can be cDNA corresponding to an RNA sequence. The template can be DNA.

As used herein, “amplification” of a template nucleic acid generally refers to a process of creating (e.g., in vitro) nucleic acid strands that are identical or complementary to at least a portion of a template nucleic acid sequence, or a universal or tag sequence that serves as a surrogate for the template nucleic acid sequence, all of which are only made if the template nucleic acid is present in a sample. Typically, nucleic acid amplification uses one or more nucleic acid polymerase and/or transcriptase enzymes to produce multiple copies of a template nucleic acid or fragments thereof, or of a sequence complementary to the template nucleic acid or fragments thereof. In vitro nucleic acid amplification techniques are may include transcription-associated amplification methods, such as Transcription-Mediated Amplification (TMA) or Nucleic Acid Sequence-Based Amplification (NASBA), and other methods such as Polymerase Chain Reaction (PCR), Reverse Transcriptase-PCR (RT-PCR), Replicase Mediated Amplification, and Ligase Chain Reaction (LCR).

In addition to those above, a wide variety of other features can be used to form the arrays described herein. For example, in some embodiments, features that are formed from polymers and/or biopolymers that are jet printed, screen printed, or electrostatically deposited on a substrate can be used to form arrays.

### EXAMPLES

The following examples are included for illustrative purposes only and are not intended to limit the scope of the present disclosure.

### Example 1: Generation of an Oligonucleotide Array with High Barcode Diversity Using Photo-Caged Hybridization/Ligation

To prepare a high-density, two-dimensional array of caged oligonucleotides, oligonucleotides of identical sequence are synthesized, and a photolabile protecting group is attached to the 3′-hydroxyl group of each oligonucleotide. The photolabile protecting groups can be removed with 365 nm light. The oligonucleotides are then immobilized via their 5′ ends to a flat glass surface in order to form a 7×7 mm array.

For uncaging, the array is first shielded with a photolithography mask before being irradiated with 365 nm light for one to ten minutes (e.g., for one to five minutes). In some examples, the array is irradiated with a total light dose of about 1-10 mW/mm². The photolithography mask has a pre-determined pattern of opaque and transparent areas such that while most of the array is shielded from light, small portions of the array remain exposed. In this manner, the photolabile protecting group is removed only from exposed portions of the array.

To add barcode sequences, the array is contacted with oligonucleotide cassettes each comprising a known barcode sequence partially hybridized to a splint. The 3′ regions of the splint are hybridized to the 5′ regions of the barcode sequences, and the 5′ regions of the splint are complementary to the 3′ regions of the immobilized oligonucleotides. Upon contact, the oligonucleotide cassettes hybridize to the immobilized oligonucleotides, to which the barcode sequences are then ligated. Ligation is only successful for immobilized oligonucleotides lacking the photolabile protecting group, leading to barcode sequences being added to only a portion of the array. Unligated oligonucleotide cassettes are subsequently removed (e.g., via nuclease digestion, capping or washing).

The above process is repeated using 125 different photolithography masks and barcode sequences until immobilized oligonucleotides in regions of interest receive a barcode sequence. By using a high number of different photolithography masks, each of which exposes only a small portion of the array to light, as well as a high number of different barcode sequences, high barcode diversity across the array is achieved. Capture sequences (e.g., poly-dT sequence) are then ligated to the barcode sequences.

### Example 2: Photo-Caged Hybridization/Ligation without Photomasking

An array of caged oligonucleotides is prepared as described in Example 1. For uncaging, a focused laser is used to irradiate a small portion of the array. The laser is sufficiently focused and the distance (pitch) between features is sufficient to prevent the laser from deprotecting groups of an adjacent feature on the array. For example, a photolithography mask may not be needed to shield other portions of the array when the feature size is large (e.g., >30 microns). Barcode sequences are then added as described in Example 1. Additional uncaging and ligation steps using different photolithography masks and barcode sequences are performed until immobilized oligonucleotides in regions of interests receive a barcode sequence.

### Example 3: Photo-Caged Hybridization/Ligation with Removal of Unligated Oligonucleotides

After each barcode sequence ligation step, any uncaged oligonucleotides to which barcode sequences are not successfully ligated are removed.

A. Nuclease Digestion

An array is prepared, uncaged, and contacted with oligonucleotide cassettes as described in Example 1. After ligation of barcode sequences, the array is contacted with exonuclease I in order to digest any uncaged oligonucleotides to which barcode sequences were not successfully ligated. After digestion, additional uncaging and ligation steps using different photolithography masks and barcode sequences are performed until immobilized oligonucleotides in regions of interest receive a barcode sequence.

B. 3′-Hydroxyl Group Capping

An array is prepared, uncaged, and contacted with oligonucleotide cassettes as described in Example 1. After ligation of barcode sequences, A-tailing with a terminal transferase is performed in order to cap any uncaged oligonucleotides to which barcode sequences were not successfully ligated, thus preventing any future ligation for the capped oligonucleotides. After digestion, additional uncaging and ligation steps using different photolithography masks and barcode sequences are performed until immobilized oligonucleotides in regions of interest receive a barcode sequence.

C. Nuclease Digestion with the Use of Nuclease-Resistant Barcodes

An array is prepared and uncaged as described in Example 1. The array is then contacted with oligonucleotide cassettes containing nuclease-resistant barcode sequences. After a round 1 barcode sequence ligation, the array is contacted with a nuclease in order to digest all oligonucleotides that lack a barcode sequence. A universal round 1 adaptor is then attached to the nuclease-resistant barcode. The universal round 1 adaptor comprises a photolabile protecting group that inhibits or prevents hybridization and/or ligation until photo cleavage.

### Example 4: Photo-Caged Hybridization/Ligation Using Alternative Caning Approaches

Alternative oligonucleotide caging approaches are used.

A. Caged Nucleobases

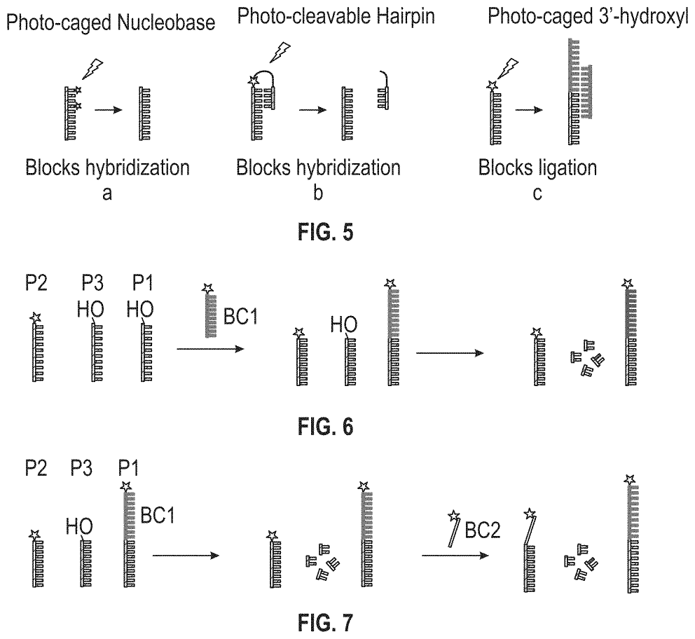

During oligonucleotide synthesis, caged deoxythymines (dT) bearing photolabile protecting groups are incorporated into each oligonucleotide, after which a high-density, 7×7 mm array is prepared. As described in Example 1, portions of the array are uncaged using 365 nm light, as shown in FIG. 5a, and the array is contacted with oligonucleotide cassettes. Hybridization is only successful for immobilized oligonucleotides from which the photolabile protecting groups have been removed. Additional uncaging and hybridization steps using different photolithography masks and barcode sequences are performed until an oligonucleotide cassette is hybridized to each immobilized oligonucleotide, to which all barcode sequences are then ligated.

B. Photocleavable Hairpin

After oligonucleotide synthesis, a photocleavable linker connected to a blocker sequence is added to the 3′ end of each oligonucleotide. The blocker sequences are complementary to the 3′ regions of the oligonucleotides and hybridize upon being added, as shown in FIG. 5b. A high-density, 7×7 mm array is prepared, and portions of the array are uncaged as described in Example 1. In this case, irradiation leads to the cleaving of exposed linkers. After irradiation, cleaved linkers and associated blocker sequences are removed, and the array is contacted with oligonucleotide cassettes. Hybridization is only successful for immobilized oligonucleotides from which the blocker sequences have been removed. Additional uncaging and hybridization steps using different photolithography masks and barcode sequences are performed until an oligonucleotide cassette is hybridized to each immobilized oligonucleotide, to which all barcode sequences are then ligated.

C. Photo-Caged 3-Hydroxyl Group

A high-density, 7×7 mm array is prepared, and each oligonucleotide is immobilized to the array substrate at the 5′ end while the 3′ end comprises a photo-caged 3-hydroxyl group. Portions of the array are uncaged as described in Example 1. In this case, irradiation leads to the exposure of the protected 3-hydroxyl group. After irradiation, the array is contacted with oligonucleotide cassettes, as shown in FIG. 5c. Additional uncaging and hybridization steps using different photolithography masks and barcode sequences are performed until an oligonucleotide cassette is hybridized to each immobilized oligonucleotide, to which all barcode sequences are then ligated.

### Example 5: Photo-Caged Hybridization/Ligation Using Combinations of Caging Approaches

Combinations of various oligonucleotide caging approaches are used.

A. Combining Photolabile Protecting Groups Removable by Different Lights

A high-density, 7×7 mm array of caged oligonucleotides is prepared. Half of the oligonucleotides can be uncaged with 254 nm light, and the other half can be uncaged with 365 nm light. After being shielded with a photolithography mask, the array is irradiated first with 254 nm light. After introduction of oligonucleotide cassettes and ligation of barcode sequences, the array is next irradiated with 365 nm light, and different barcode sequences are introduced and ligated. Additional uncaging and ligation steps using different photolithography masks and barcode sequences are performed using both 254 nm and 365 nm light until immobilized oligonucleotides in regions of interest receive a barcode sequence.

B. Uncaging Nucleobases and 3′-Hydroxyl Groups Using Different Lights

A high-density, 7×7 mm array of caged oligonucleotides is prepared. Half of the oligonucleotides contain caged 3′-hydroxyl groups that can be uncaged with 365 nm light, and the other half contain caged dTs that can be uncaged with 254 nm light. After being shielded with a photolithography mask, the array is irradiated first with 365 nm light. Oligonucleotide cassettes are introduced and hybridize to immobilized oligonucleotides with previously caged 3′-hydroxyl groups. The array is next irradiated with 254 nm light. Different oligonucleotide cassettes are then introduced to hybridize to oligonucleotides with previously caged dTs, after which barcode sequences from all hybridized oligonucleotide cassettes are ligated. Additional uncaging and hybridization/ligation steps using different photolithography masks and barcode sequences are performed using both 254 nm and 365 nm light until immobilized oligonucleotides in regions of interest receive a barcode sequence.

C. Uncaging Nucleobases and 3′-Hydroxyl Groups Using the Same Light

A high-density, 7×7 mm array of caged oligonucleotides is prepared. Half of the oligonucleotides contain caged dTs, and the other half contain caged 3′hydroxyl groups. All oligonucleotides can be uncaged with 365 nm light. Without irradiation, oligonucleotide cassettes are introduced and hybridize to immobilized oligonucleotides with caged 3′-hydroxyl groups. After hybridization, a photolithography mask is applied, and the array is irradiated with 365 nm light. Different oligonucleotide cassettes are then introduced to hybridize to oligonucleotides with previously caged dTs, after which barcode sequences from all hybridized oligonucleotide cassettes are ligated. Additional uncaging and hybridization/ligation steps using different photolithography masks and barcode sequences are performed until immobilized oligonucleotides in regions of interest receive a barcode sequence.

### Example 6: Increasing Barcode Diversity Using Multiple Rounds of Photo-Caged Hybridization/Ligation

Multiple rounds of photo-caged hybridization/ligation are used to increase barcode diversity.

A. Adding a Common Barcode Sequence During the First Round

A 10-inch wafer with 125 7×7 mm arrays is prepared. All oligonucleotides contain a caged 3′-hydroxyl group. During the first round, all oligonucleotides are simultaneously uncaged (i.e., without the use of a photolithography mask), and a common barcode sequence with a caged 3′-hydroxyl group is ligated to all oligonucleotides. Two subsequent rounds are performed as described in Example 1, each round using 125 different photolithography masks and caged barcode sequences. In these two rounds, irradiation leads to the uncaging of previously added barcode sequences, to which subsequent barcode sequences are ligated. After the last round, all barcode sequences are simultaneously uncaged, and capture sequences are added and ligated.

B. Adding Unique Barcode Sequences During Each Round

A 10-inch wafer with 125 7×7 mm arrays is prepared. All oligonucleotides contain a caged 3′-hydroxyl group. Three rounds are performed as described in Example 1, each round using 125 different photolithography masks and barcode sequences with caged 3′-hydroxyl groups. After the last round, all barcode sequences are simultaneously uncaged, and capture sequences are added and ligated. In this manner, 1,963,125 unique barcodes per array are generated after three rounds, resulting in arrays of five-micron resolution. A wafer is prepared in under five hours.

C. Adding Barcode Sequences with Different Photolabile Protecting Groups

A 10-inch wafer with 125 7×7 mm arrays is prepared. All oligonucleotides contain a caged 3′-hydroxyl group. Three rounds are performed as described in Example 1, each round using 125 different photolithography masks and barcode sequences containing caged dTs. After the last round, all barcode sequences are simultaneously uncaged, and capture sequences are added and ligated.

D. Adding Photolabile Protecting Groups after Barcode Sequence Ligation

A 10-inch wafer with 125 7×7 mm arrays is prepared. All oligonucleotides contain a caged 3′-hydroxyl group. Three rounds are performed as described in Example 1, each round using 125 different photolithography masks and non-caged barcode sequences. At the end of each round, an adapter sequence with a caged 3′-hydroxyl group is added and ligated to each of the barcode sequences added during that round. After the last round, capture sequences are added and ligated.

### Example 7: Multiple Rounds of Photo-Caged Hybridization/Ligation with Removal of Unligated Oligonucleotides

A 7×7 mm array of caged oligonucleotides is prepared and uncaged as described in Example 1. After uncaging, the array is contacted with oligonucleotide cassettes containing nuclease-resistant, non-caged barcode sequences. After barcode sequence ligation, the array is contacted with a nuclease to digest all oligonucleotides not ligated to a barcode sequence. After digestion, an adapter sequence with a caged 3′-hydroxyl group is ligated to the remaining oligonucleotides. Additional uncaging, ligation, and digestion steps are performed.

### Example 8: Increasing Barcode Diversity by Pre-Patterning the Oligonucleotide Array Prior to Photo-Caged Hybridization/Ligation

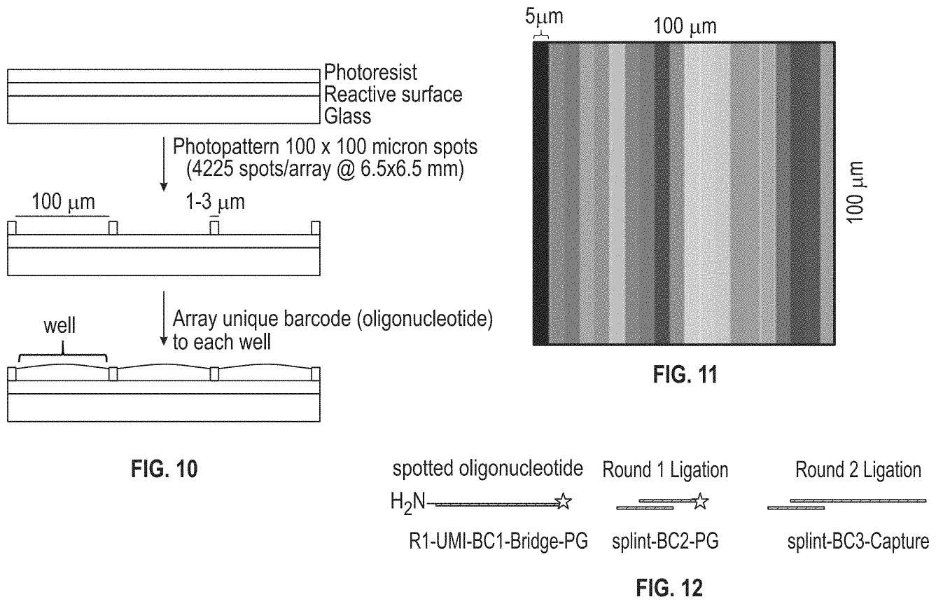

Positive photoresist exposure and development is used to pre-pattern an oligonucleotide array. As shown in FIG. 10, a positive photoresist is applied to a glass surface, and a patterned mask is used to block portions of the photoresist from light. After application of a developer solvent and irradiation with light, exposed portions of the photoresist are degraded such that 4225 100×100 micron wells per 6.5×6.5 mm array are formed. Wells are spaced one to three microns apart. Caged oligonucleotides are then immobilized to the glass surface at the bottom of each well, each well receiving a unique oligonucleotide sequence.

A. Adding a Common Barcode Sequence

Without the use of a photolithography mask, all caged oligonucleotides of a pre-patterned array are unmasked, and a common barcode sequence is added to each oligonucleotide. In this approach, barcode diversity stems solely from initial pre-patterning of the array with diverse sets of oligonucleotides.

B. Adding Unique Barcode Sequences During Multiple Rounds

In another approach, all wells of a pre-patterned array are uncaged as described in Example 1. For each well, one 100×5 micron segment is uncaged at a time, for example, as shown in FIG. 11, leading to 20 different barcode sequences being added to each well during the first round. The second round proceeds similarly, though with segments perpendicular to those of the first round. The addition of 20 more different barcode sequences during the second round results in a total of 400 features per well after two rounds.

An exemplary barcode assembly scheme is shown in FIG. 12. The spotted oligo comprises R1-UMI-BC1-Bridge-PG, wherein R1 is a read1 sequence (e.g., serving as a sequencing primer or PCR handle), UMI is a unique molecular identifier, BC1 is a barcode, Bridge is a bridge sequence, and PG is a photolabile protective group. For round 1 ligation, the splint-BC2-PG oligo is used, where splint is a sequence that hybridizes to the bridge sequence upon deprotection of the spotted oligo, BC2 is a barcode, and PG is a photolabile protective group. For round 2 ligation, the splint-BC3-Capture oligo is used, where splint is a sequence that hybridizes to the splint sequence in the round 1 ligation product, BC3 is a barcode, and Capture is a capture sequence.

### Example 9: Generation of an Oligonucleotide Array with High Barcode Diversity Using Photo-caged Templated DNA Synthesis

Instead of using oligonucleotide cassettes containing pre-synthesized barcode sequences, barcode sequences are synthesized in situ using splints as templates.

A. One Round of Templated DNA Synthesis

An array of oligonucleotides with caged dTs is prepared. After uncaging as described in Example 1, splints with 5′ regions complementary to the 3′ regions of the immobilized oligonucleotides are added. After hybridization, barcode sequences are synthesized in situ using the unhybridized portions of the splints as templates. Newly synthesized barcode sequences are then ligated to the immobilized oligonucleotides. Additional uncaging, synthesis, and ligation steps using different photolithography masks and barcode sequences are performed until immobilized oligonucleotides in regions of interest receive a barcode sequence.

B. Multiple Rounds of Templated DNA Synthesis

An array of oligonucleotides with caged dTs is prepared. After uncaging as described in Example 1, splints with 5′ regions complementary to the 3′ regions of the immobilized oligonucleotides are added. After hybridization, barcode sequences are synthesized in situ using the unhybridized portions of the splints as templates, during which time caged dTs are incorporated into the barcode sequences. The barcode sequences are then ligated to the immobilized oligonucleotides. Additional uncaging, synthesis, and ligation steps using different photolithography masks and barcode sequences are performed until immobilized oligonucleotides in regions of interest receive a barcode sequence. Two additional rounds are performed such that additional barcode sequences are synthesized and ligated to previously added barcodes. After three rounds of templated DNA synthesis are complete, all barcode sequences are uncaged, and capture sequences are added and ligated.

### Example 10: Irradiation-Dependent Ligation of a Photo-Caged Oligonucleotide

The present example demonstrates ligation of a photoprotected oligonucleotide to a base oligonucleotide (e.g., a common R1 primer sequence) immobilized on a substrate (e.g., a glass slide), wherein the ligation depends on removal of a photoprotective group (PPG) from the photoprotected oligonucleotide.

Oligonucleotides were printed in wells on a coated substrate (e.g., a glass slide) to create a base layer of unprotected oligos. First, oligonucleotides were spotted in wells in printing buffer (sodium phosphage buffer at pH 8.5 with a surfactant, such as 0.06% sarcosyl). After drying the oligonucleotide spots at room temperature, the slide was incubated at >75% Relative Humidity for about 3-18 hours. Slides were then incubated with blocking and wash buffers according to standard protocols and dried to generate the substrate with immobilized base oligonucleotides. The immobilized oligonucleotides included a first primer sequence (R1).

Different portions of the substrate were exposed to a 5′ photoprotected (PPG) oligonucleotide and ligation mix (which includes a ligase and splint oligonucleotide) with or without irradiation to remove the PPG. In this example, the PPG was a photo-cleavable spacer comprising the following structure:

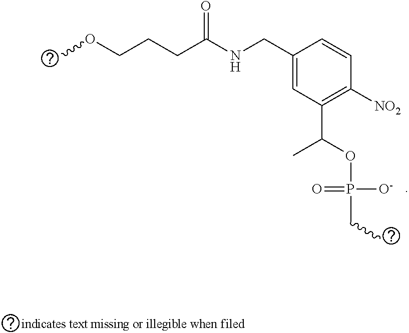

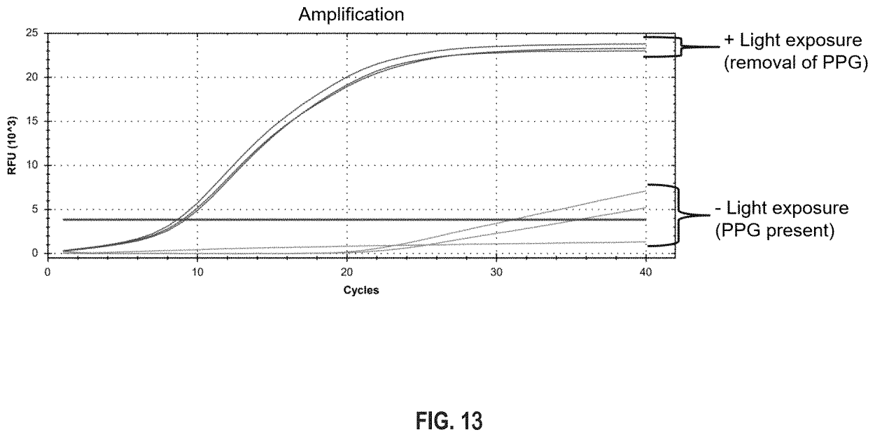

Cleavage of the PPG revealed a free 5′ phosphate, allowing the splint to facilitate ligation of the PPG oligonucleotide to the base oligonucleotide. The ligation reaction also introduced a second primer sequence which was used for a qPCR assay described below. After ligation occurs, second strand synthesis was performed using a second strand reagent and a template switch oligo and denaturation of the second strand from the substrate. Finally, a TaqMan quantitative PCR (qPCR) assay was performed to detect successful ligation of the PPG oligonucleotide to the base oligonucleotide. As shown in FIG. 13, irradiation with light resulted in removal of the PPG and ligation of the PPG oligonucleotide containing the second primer sequence to the base oligonucleotide (which led to qPCR amplification and detection of ligated product), whereas the presence of the PPG (non-irradiated portion) prevented ligation (which led to no detection of the ligated product by qPCR).

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method for providing an array of polynucleotides, comprising:
irradiating a first polynucleotide immobilized on a substrate with a first light while a second polynucleotide immobilized on the substrate is not irradiated with the first light, wherein the first polynucleotide comprises a first photo-cleavable moiety that inhibits or blocks hybridization and/or ligation to the first polynucleotide, and the second polynucleotide comprises a second photo-cleavable moiety that inhibits or blocks hybridization and/or ligation to the second polynucleotide,
thereby cleaving the first photo-cleavable moiety such that the inhibition or blocking of hybridization and/or ligation to the first polynucleotide is reduced or eliminated, whereas hybridization and/or ligation to the second polynucleotide remain inhibited or blocked by the second photo-cleavable moiety,
wherein a first barcode is attached to the first polynucleotide via hybridization and/or ligation,
thereby providing on the substrate an array comprising the first and second polynucleotides, wherein the first polynucleotide is barcoded with the first barcode and the second polynucleotide is not barcoded with the first barcode.

2-3. (canceled)

4. The method of claim 1, further comprising irradiating the second polynucleotide with a second light, thereby cleaving the second photo-cleavable moiety such that the inhibition or blocking of hybridization and/or ligation to the second polynucleotide is reduced or eliminated.

5. The method of claim 4, wherein the second polynucleotide is irradiated with the second light while the first polynucleotide is not irradiated with the second light.

6. (canceled)

7. The method of claim 1, further comprising attaching a second barcode to the second polynucleotide via hybridization and/or ligation, thereby providing on the substrate an array comprising the first polynucleotide barcoded with the first barcode and the second polynucleotide barcoded with the second barcode.

8-18. (canceled)

19. The method of claim 1, wherein hybridization and/or ligation to the first polynucleotide barcoded with the first barcode is inhibited or blocked, and/or
wherein hybridization and/or ligation to the second polynucleotide barcoded with the second barcode is inhibited or blocked.

20. The method of claim 1, wherein the first barcode comprises a third photo-cleavable moiety that inhibits or blocks hybridization and/or ligation, thereby inhibiting or blocking hybridization and/or ligation to the first polynucleotide barcoded with the first barcode.

21-24. (canceled)

25. The method of claim 1 wherein the first photo-cleavable moiety and/or the second photo-cleavable moiety comprise a photo-caged nucleobase.

26. The method of claim 1, wherein the first photo-cleavable moiety and/or the second photo-cleavable moiety comprise a photo-cleavable hairpin.

27. The method of claim 1, wherein the first photo-cleavable moiety and/or the second photo-cleavable moiety comprise a photo-caged 3′-hydroxyl group.

28-34. (canceled)

35. The method of claim 1, wherein the substrate comprises a plurality of differentially barcoded polynucleotides immobilized thereon.

36. (canceled)

37. The method of claim 1, wherein irradiating the sample comprises using a photomask to selectively irradiate the first polynucleotide or the second polynucleotide.

38. The method of claim 1, wherein the attachment of the first barcode comprises ligating one end of the first barcode to one end of the first polynucleotide.

39-40. (canceled)

41. The method of claim 1, wherein the attachment of the first barcode comprises hybridizing one end of the first barcode and one end of the first polynucleotide to a first splint.

42. The method of claim 41, further comprising ligating the first barcode to the first polynucleotide hybridized to the first splint.

43. (canceled)

44. The method of claim 42, wherein ligating the first barcode to the first polynucleotide, respectively, is preceded by gap filling.

45-49. (canceled)

50. A method for providing an array of polynucleotides, comprising:
(a1) irradiating polynucleotide P1 immobilized on a substrate with light while polynucleotide P2 immobilized on the substrate is photomasked, wherein polynucleotides P1 and P2 comprise a photo-cleavable moiety that inhibits or blocks hybridization and/or ligation to P1 and P2, respectively,
thereby cleaving the photo-cleavable moiety to allow hybridization and/or ligation to P1, whereas hybridization and/or ligation to P2 remain inhibited or blocked by the photo-cleavable moiety; and
(b1) attaching barcode 1A to P1 via hybridization and/or ligation to form a barcoded polynucleotide 1A-P1, wherein barcode 1A comprises the photo-cleavable moiety which inhibits or blocks hybridization and/or ligation to 1A-P1,
thereby providing on the substrate an array comprising polynucleotides 1A-P1 and P2 each comprising the photo-cleavable moiety that inhibits or blocks hybridization and/or ligation.

51. The method of claim 50, further comprising:
(c1) irradiating P2 with light while 1A-P1 is photomasked, thereby cleaving the photo-cleavable moiety to allow hybridization and/or ligation to P2, whereas hybridization and/or ligation to 1A-P1 remain inhibited or blocked by the photo-cleavable moiety; and
(d1) attaching barcode 1B to P2 via hybridization and/or ligation to form a barcoded polynucleotide 1B-P2, wherein barcode 1B comprises the photo-cleavable moiety which inhibits or blocks hybridization and/or ligation to 1B-P2,
thereby providing on the substrate an array comprising barcoded polynucleotides 1A-P1 and 1B-P2 each comprising the photo-cleavable moiety that inhibits or blocks hybridization and/or ligation.

52-53. (canceled)

54. The method of claim 51, further comprising:
(a2) irradiating one of 1A-P1 and 1B-P2 with light while the other is photomasked,
thereby cleaving the photo-cleavable moiety to allow hybridization and/or ligation to the irradiated polynucleotide, whereas hybridization and/or ligation to the photomasked polynucleotide remains inhibited or blocked by the photo-cleavable moiety; and
(b2) attaching barcode 2A to the irradiated polynucleotide via hybridization and/or ligation to form a 2A-barcoded polynucleotide, wherein barcode 2A comprises the photo-cleavable moiety which inhibits or blocks hybridization and/or ligation,
thereby providing on the substrate an array comprising barcoded polynucleotides each comprising the photo-cleavable moiety that inhibits or blocks hybridization and/or ligation.

55. The method of claim 54, further comprising:
(c2) irradiating the photomasked polynucleotide in step a2 with light while the 2A-barcoded polynucleotide is photomasked, thereby cleaving the photo-cleavable moiety to allow hybridization and/or ligation, whereas hybridization and/or ligation to the 2A-barcoded polynucleotide remain inhibited or blocked by the photo-cleavable moiety; and
(d2) attaching barcode 2B to the irradiated polynucleotide in step c2 via hybridization and/or ligation to form a 2B-barcoded polynucleotide, wherein barcode 2B comprises the photo-cleavable moiety which inhibits or blocks hybridization and/or ligation,
thereby providing on the substrate an array comprising barcoded polynucleotides each comprising the photo-cleavable moiety that inhibits or blocks hybridization and/or ligation.

56-57. (canceled)

58. The method of claim 55, wherein steps a1-d1 form round 1 and steps a2-d2 form round 2, the method further comprising steps ai-di in round i, wherein barcodes iA and iB are attached to provide barcoded polynucleotides on the substrate, and wherein i is an integer greater than 2.

59-64. (canceled)

65. The method of claim 50, wherein the photo-cleavable moiety comprises a photo-caged nucleobase.

66. The method of claim 65, wherein the photo-caged nucleobase is a photo-caged deoxythymidine (dT).

67. The method of claim 65, wherein the photo-cleavable moiety comprises the following structure:

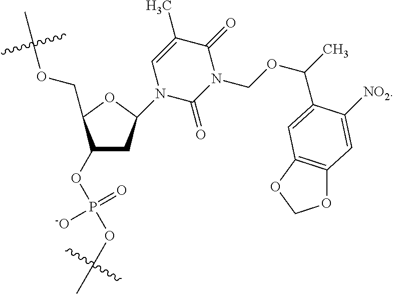

68. The method of claim 50, wherein the photo-cleavable moiety comprises a photo-cleavable hairpin.

69. The method of claim 68, wherein the photo-cleavable moiety comprises the following structure:

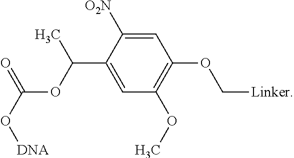

70. The method of claim 50, wherein the photo-cleavable moiety comprises a photo-caged 3-hydroxyl group.

71. The method of claim 70, wherein the photo-cleavable moiety comprises the following structure:

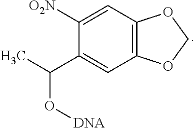

72-85. (canceled)

86. The method of claim 1, wherein polynucleotides of different nucleic acid sequences are immobilized on the substrate in a pattern comprising rows and columns prior to the irradiation.

87-118. (canceled)

119. A hybridization complex, comprising:
a first polynucleotide immobilized on a substrate;
a first splint hybridized to one end of the first polynucleotide and one end of a first barcode; and
the first barcode which comprise a first photo-cleavable moiety that inhibits or blocks hybridization and/or ligation to the other end of the first barcode.

120-138. (canceled)
